(19) United States
(12) Patent Application Publication
(10) Pub. No.: US 2022/0323427 A1
(43) Pub. Date: Oct. 13, 2022

(54) SOMATOSTATIN RECEPTOR TYPE 5 AGONIST FOR THE TREATMENT OF HYPERINSULINISM

(71) Applicant: Crinetics Pharmaceuticals, Inc., San Diego, CA (US)

(72) Inventors: Christine FERRARA-COOK, San Diego, CA (US); Alan S. KRASNER, San Diego, CA (US)

(21) Appl. No.: 17/673,588

(22) Filed: Feb. 16, 2022

Related U.S. Application Data
(60) Provisional application No. 63/150,266, filed on Feb. 17, 2021.
(60) Provisional application No. 63/244,039, filed on Sep. 14, 2021.

(51) Int. Cl. A61K 31/4439; A61P 3/08

(52) U.S. Cl. CPC A61K 31/4439; A61P 3/08

(57) ABSTRACT

Provided herein are methods and compositions for the treatment of hyperinsulinism

### CROSS-REFERENCE TO RELATED APPLICATIONS

This application claims the benefit of U.S. Provisional Application No. 63/150,266 filed on Feb. 17, 2021, and U.S. Provisional Application No. 63/244,039 filed on Sep. 14, 2021, each of which is incorporated herein by reference in its entirety.

### FIELD OF THE INVENTION

Described herein is a somatostatin receptor type 5 (SST5) agonist and methods of using the STT5 agonist in the treatment of conditions, diseases, or disorders that would benefit from modulating somatostatin receptor type 5 activity, such as hyperinsulinism.

### BACKGROUND OF THE INVENTION

Congenital hyperinsulinism (HI) is a rare disease resulting in excess insulin secretion from the pancreatic β-cells even when blood glucose levels are low, leading to life-threatening hypoglycemia. Patients with congenital HI are vulnerable to permanent brain injury and death. Current treatments are limited and not universally effective in all patients. Provided herein are methods and compositions for the treatment of congenital HI.

### SUMMARY OF THE INVENTION

Provided herein are methods for the treatment of HI.

In one aspect, described herein is a method of treating hyperinsulinism (HI) in a human, the method comprising administering to the human in need thereof a compound having the structure of Compound 1, or a pharmaceutically acceptable salt, or solvate thereof:

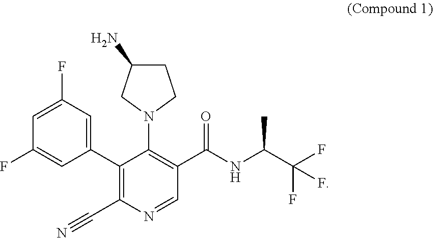 [SMILES: C[C@H](NC(=O)c1cnc(C#N)c(-c2cc(F)cc(F)c2)c1N1CC[C@H](N)C1)C(F)(F)F]

In some embodiments, the hyperinsulinism comprises hyperinsulinemic hypoglycemia. In some embodiments, the hyperinsulinism comprises congenital hyperinsulinism.

In some embodiments, the human comprises at least one mutation in the adenosine triphosphate-dependent potassium (K_{ATP}) channel. In some embodiments, the human comprises at least one mutation or defect in the ABCC8 and KCNJ11 genes that encode the SUR-1 and Kir6.2 (potassium channel) subunits of the K_{ATP} channel, glutamate dehydrogenase (GLUD1), glucokinase (GCK), hepatic nuclear transcription factor 4A (HNF4A), hepatic nuclear transcription factor 1A (HNF1A), hexokinase (HK1), uncoupling protein 2 (UCP2), short-chain 3-OH acyl-CoA dehydrogenase (HADH), solute carrier family 16 member 1 (SLC16A1), monocarboxylate transporter 1 (MCT1), or a combination thereof. In some embodiments, the congenital hyperinsulinism comprises transient hyperinsulinism, focal hyperinsulinism, or diffuse hyperinsulinism. In some embodiments, the congenital hyperinsulinism comprises glucokinase gain-of-function mutations, hyperammonemic hyperinsulinism (glutamate dehydrogenase gain-of-function mutations), short chain acyl coenzyme A dehydrogenase deficiency, carbohydrate-deficient glycoprotein syndrome (Jaeken's Disease), or Beckwith-Wiedemann syndrome. In some embodiments, the hyperinsulinism comprises diazoxide-unresponsive hyperinsulinism. In some embodiments, the hyperinsulinism is acquired hyperinsulinism. In some embodiments, the acquired hyperinsulinism comprises pancreatic insulinoma, nesidioblastosis, drug induced hyperinsulinism, or combinations thereof.

In some embodiments, the human is less than 12 years old, less than 6 years old, less than 4 years old, less than 3 years old, less than 2 years old, less than 1.5 years old, less than 1 year old, or less than 6 months old.

In some embodiments, treating hyperinsulinism comprises increasing levels of plasma glucose, β-hydroxybutyrate, glucagon, or a combination thereof. In some embodiments, treating hyperinsulinism comprises reducing plasma levels of: insulin, C peptide, or a combination thereof. In some embodiments, treating hyperinsulinism comprises reducing plasma insulin levels. In some embodiments, reducing plasma levels of insulin comprise reducing levels of incretin-induced insulin secretion. In some embodiments, treating hyperinsulinism comprises increasing plasma glucose to the average level of a subject without hyperinsulinism. In some embodiments, treating hyperinsulinism comprises maintaining plasma glucose levels at above at least 50 mg/dL, above at least 60 mg/dL, above at least 70 mg/dL, or above at least 80 mg/dL. In some embodiments, treating hyperinsulinism comprises reducing insulin secretion from pancreatic β-cells. In some embodiments, treating hyperinsulinism comprises decreasing or inhibiting insulin secretion and minimizing or avoiding glucagon suppression. In some embodiments, treating hyperinsulinism comprises reducing the risk of brain damage, reducing the extent of brain damage, reducing the risk of pancreatectomy, or a combination thereof. In some embodiments, treating hyperinsulinism comprises reducing hypoketotic hypoglycemia, reducing lethargy, reducing irritability, reducing reducing the risk of vision loss, reducing the risk of neurocognitive defects, reducing the risk of seizures, reducing the risk of apnea, reducing the risk of coma, reducing the risk of death, or a combination thereof.

In some embodiments, Compound 1, or a pharmaceutically acceptable salt, or solvate thereof, is administered daily. In some embodiments, Compound 1, or a pharmaceutically acceptable salt, or solvate thereof, is administered once daily or twice daily. In some embodiments, Compound 1, or a pharmaceutically acceptable salt, or solvate thereof, is administered orally.

In some embodiments, Compound 1, or a pharmaceutically acceptable salt, or solvate thereof, is administered in amount equivalent to about 0.05 mg to about 200 mg of Compound 1. In some embodiments, Compound 1, or a pharmaceutically acceptable salt, or solvate thereof, is administered in amount equivalent to about 0.5 mg to about 100 mg of Compound 1. In some embodiments, Compound 1, or a pharmaceutically acceptable salt, or solvate thereof, is administered in amount equivalent to about: 0.05 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13.0 mg, 13.5 mg, 14.0 mg, 14.5 mg, 15.0 mg, 15.5 mg, 16.0 mg, 16.5 mg, 17.0 mg, 17.5 mg, 18.0 mg, 18.5 mg, 19.0 mg, 19.5 mg, or 20.0 mg of Compound 1.

In some embodiments, Compound 1, or a pharmaceutically acceptable salt, or solvate thereof, is administered in a daily dosage equivalent to about 0.01 mg/kg to about 50 mg/kg of Compound 1. In some embodiments, Compound 1, or a pharmaceutically acceptable salt, or solvate thereof, is administered in a daily dosage equivalent to about 0.1 mg/kg to about 5.0 mg/kg of Compound 1. In some embodiments, Compound 1, or a pharmaceutically acceptable salt, or solvate thereof, is administered in a daily dosage equivalent to about: 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.25 mg/kg, 2.5 mg/kg, 2.75 mg/kg, 3.0 mg/kg, 3.25 mg/kg, 3.5 mg, 3.75 mg/kg, 4.0 mg/kg, 4.25 mg/kg, 4.5 mg/kg, 4.75 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, or 10.0 mg/kg of Compound 1.

In some embodiments, Compound 1, or a pharmaceutically acceptable salt, or solvate thereof, is administered daily in an amount sufficient to maintain plasma glucose levels at above at least 50 mg/dL, above at least 60 mg/dL, above at least 70 mg/dL, or above at least 80 mg/dL over at least 12 hours, at least 18 hours, or at least 24 hours.

In another aspect, described herein is a method of treating congenital hyperinsulinism in a human, the method comprising administering to the human in need thereof, a compound having the structure of Compound 1, or a pharmaceutically acceptable salt, or solvate thereof. In some embodiments, the human comprises at least one mutation or defect in the adenosine triphosphate-dependent potassium (K_{ATP}) channel. In some embodiments, the human comprises at least one mutation or defect in the ABCC8 and KCNJ11 genes that encode the SUR-1 and Kir6.2 (potassium channel) subunits of the K_{ATP} channel, glutamate dehydrogenase (GLUD1), glucokinase (GCK), hepatic nuclear transcription factor 4A (HNF4A), hepatic nuclear transcription factor 1A (HNF1A), hexokinase (HK1), uncoupling protein 2 (UCP2), short-chain 3-OH acyl-CoA dehydrogenase (HADH), solute carrier family 16 member 1 (SLC16A1), monocarboxylate transporter 1 (MCT1), or a combination thereof. In some embodiments, congenital hyperinsulinism comprises transient hyperinsulinism, focal hyperinsulinism, or diffuse hyperinsulinism. In some embodiments, congenital hyperinsulinism comprises glucokinase gain-of-function mutations, hyperammonemic hyperinsulinism (glutamate dehydrogenase gain-of-function mutations), short chain acyl coenzyme A dehydrogenase deficiency, carbohydrate-deficient glycoprotein syndrome (Jaeken's Disease), or Beckwith-Wiedemann syndrome. In some embodiments, congenital hyperinsulinism comprises diazoxide-unresponsive congenital hyperinsulinism. In some embodiments, the human is less than 12 years old, less than 6 years old, less than 4 years old, less than 3 years old, less than 2 years old, less than 1.5 years old, less than 1 year old, or less than 6 months old.

In some embodiments, treating congenital hyperinsulinism comprises increasing levels of plasma glucose, β-hydroxybutyrate, glucagon, or a combination thereof. In some embodiments, treating congenital hyperinsulinism comprises reducing plasma levels of: insulin, C peptide, or a combination thereof. In some embodiments, treating congenital hyperinsulinism comprises reducing plasma insulin levels. In some embodiments, reducing plasma levels of insulin comprise reducing levels of incretin-induced insulin secretion. In some embodiments, treating congenital hyperinsulinism comprises increasing plasma glucose to the average level of a subject without congenital hyperinsulinism. In some embodiments, treating congenital hyperinsulinism comprises maintaining plasma glucose levels at above at least 50 mg/dL, above at least 60 mg/dL, above at least 70 mg/dL, or above at least 80 mg/dL. In some embodiments, treating congenital hyperinsulinism comprises reducing insulin secretion from pancreatic β-cells. In some embodiments, treating congenital hyperinsulinism comprises decreasing or inhibiting insulin secretion and minimizing or avoiding glucagon suppression. In some embodiments, treating congenital hyperinsulinism comprises reducing the risk of brain damage, reducing the extent of brain damage, reducing the risk of pancreatectomy, or a combination thereof. In some embodiments, treating congenital hyperinsulinism comprises reducing hypoketotic hypoglycemia, reducing lethargy, reducing irritability, reducing the risk of vision loss, reducing the risk of neurocognitive defects, reducing the risk of seizures, reducing the risk of apnea, reducing the risk of coma, reducing the risk of death, or a combination thereof.

In a further aspect, described herein is a method of inhibiting insulin secretion from the pancreas in a human with congenital hyperinsulinism comprising administering to the human in need thereof a compound having the structure of Compound 1, or a pharmaceutically acceptable salt, or solvate thereof. In some embodiments, inhibiting insulin secretion comprises inhibiting insulin secretion from pancreatic β-cells. In some embodiments, inhibiting insulin secretion comprises inhibiting incretin-induced insulin secretion. In some embodiments, inhibiting insulin secretion comprises treating recurrent hypoglycemia.

In yet another aspect, described herein is a method of decreasing insulin levels in a human with recurrent hypoglycemia, the method comprising administering to the human in need thereof, a compound having the structure of Compound 1, or a pharmaceutically acceptable salt, or solvate thereof. In some embodiments, the human with recurrent hypoglycemia comprises hyperinsulinism. In some embodiments, the human with recurrent hypoglycemia comprises hyperinsulinism. In some embodiments, reducing levels of insulin comprise reducing levels of incretin-induced insulin secretion. In some embodiments, insulin levels are decreased by reducing insulin secretion from a pancreatic β-cell.

In another aspect, described herein is a method of treating or preventing hypoglycemia in a human with hyperinsulinism, the method comprising administering to the human in need thereof, a compound having the structure of Compound 1, or a pharmaceutically acceptable salt, or solvate thereof. In some embodiments, the hyperinsulinism comprises congenital hyperinsulinism. In some embodiments, the hypoglycemia comprises recurrent hypoglycemia.

In some embodiments, treating or preventing hypoglycemia comprises reducing hypoketotic hypoglycemia, lethargy, irritability, macrosomia, vision loss, neurocognitive defects, seizures, apnea, coma, death, or a combination thereof. In some embodiments, treating or preventing the hypoglycemia comprises reducing the risk of brain damage, reducing the extent of brain damage, reducing the risk of pancreatectomy, or a combination thereof. In some embodiments, treating or preventing hypoglycemia comprises increasing levels of plasma glucose, β-hydroxybutyrate, glucagon, or a combination thereof. In some embodiments, treating or preventing hypoglycemia comprises increasing plasma glucose levels to the average level of a human without hypoglycemia. In some embodiments, treating or preventing hypoglycemia comprises maintaining plasma glucose levels at above at least 50 mg/dL, above at least 60 mg/dL, above at least 70 mg/dL, or above at least 80 mg/dL. In some embodiments, treating or preventing hypoglycemia comprises reducing the levels of: insulin, C peptide, or a combination thereof. In some embodiments, treating or preventing hypoglycemia comprises reducing plasma levels of insulin. In some embodiments, reducing plasma levels of insulin comprise reducing levels of incretin-induced insulin secretion. In some embodiments, treating or preventing the hypoglycemia comprises reducing insulin secretion from a pancreatic β-cell. In some embodiments, treating or preventing the hypoglycemia comprises decreasing or inhibiting insulin secretion and minimizing or avoiding glucagon suppression.

In some embodiments, the human comprises at least one mutation or defect in the adenosine triphosphate-dependent potassium (K_{ATP}) channel. In some embodiments, the human comprises at least one mutation or defect in the ABCC8 and KCNJ11 genes that encode the SUR-1 and Kir6.2 (potassium channel) subunits of the K_{ATP} channel, glutamate dehydrogenase (GLUD1), glucokinase (GCK), hepatic nuclear transcription factor 4A (HNF4A), hepatic nuclear transcription factor 1A (HNF1A), hexokinase (HK1), uncoupling protein 2 (UCP2), short-chain 3-OH acyl-CoA dehydrogenase (HADH), solute carrier family 16 member 1 (SLC16A1), monocarboxylate transporter 1 (MCT1), or a combination thereof. In some embodiments, the human comprises transient hyperinsulinism, focal hyperinsulinism, or diffuse hyperinsulinism. In some embodiments, the human comprises glucokinase gain-of-function mutations, hyperammonemic hyperinsulinism (glutamate dehydrogenase gain-of-function mutations), short chain acyl coenzyme A dehydrogenase deficiency, carbohydrate-deficient glycoprotein syndrome (Jaeken's Disease), or Beckwith-Wiedemann syndrome. In some embodiments, the human is less than 12 years old, less than 6 years old, less than 4 years old, less than 3 years old, less than 2 years old, less than 1.5 years old, less than 1 year old, or less than 6 months old.

In some embodiments, Compound 1, or a pharmaceutically acceptable salt, or solvate thereof, is administered daily. In some embodiments, Compound 1, or a pharmaceutically acceptable salt, or solvate thereof, is administered once daily or twice daily. In some embodiments, Compound 1, or a pharmaceutically acceptable salt, or solvate thereof, is administered orally.

In some embodiments, Compound 1, or a pharmaceutically acceptable salt, or solvate thereof, is administered in amount equivalent to about 0.5 mg to about 100 mg of Compound 1. In some embodiments, Compound 1, or a pharmaceutically acceptable salt, or solvate thereof, is administered in amount equivalent to about: 0.05 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13.0 mg, 13.5 mg, 14.0 mg, 14.5 mg, 15.0 mg, 15.5 mg, 16.0 mg, 16.5 mg, 17.0 mg, 17.5 mg, 18.0 mg, 18.5 mg, 19.0 mg, 19.5 mg, or 20.0 mg of Compound 1.

In some embodiments, Compound 1, or a pharmaceutically acceptable salt, or solvate thereof, is administered in a daily dosage equivalent to about 0.01 mg/kg to about 50 mg/kg of Compound 1. In some embodiments, Compound 1, or a pharmaceutically acceptable salt, or solvate thereof, is administered in a daily dosage equivalent to about 0.1 mg/kg to about 5.0 mg/kg of Compound 1. In some embodiments, Compound 1, or a pharmaceutically acceptable salt, or solvate thereof, is administered in a daily dosage equivalent to about: 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.25 mg/kg, 2.5 mg/kg, 2.75 mg/kg, 3.0 mg/kg, 3.25 mg/kg, 3.5 mg, 3.75 mg/kg, 4.0 mg/kg, 4.25 mg/kg, 4.5 mg/kg, 4.75 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, or 10.0 mg/kg of Compound 1.

In some embodiments, Compound 1, or a pharmaceutically acceptable salt, or solvate thereof, is administered daily in an amount sufficient to maintain plasma glucose levels at above at least 50 mg/dL, above at least 60 mg/dL, above at least 70 mg/dL, or above at least 80 mg/dL over at least 12 hours, at least 18 hours or at least 24 hours.

Articles of manufacture, which include packaging material, Compound 1, or a pharmaceutically acceptable salt thereof, within the packaging material, and a label that indicates that Compound 1, or a pharmaceutically acceptable salt thereof, is used for modulating the activity of a somatostatin receptor (e.g. somatostatin receptor type 5 (SST5)), or for the treatment, prevention or amelioration of one or more symptoms of a disease or condition that would benefit from modulation of the activity of a somatostatin receptor (e.g. somatostatin receptor type 5 (SST5)), are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE DRAWINGS

FIG. 1 shows the effect of Compound 1 on sulfonylurea-induced hypoglycemia in rats.

FIG. 2 shows the SAD study pharmacokinetic results (0.5 to 120 mg of Compound 1).

FIG. 3 shows the SAD study results of the dose response and PK/PD on pre- and post-stimulated glucose and insulin in an IVGTT (0.5 to 120 mg of Compound 1).

FIG. 4 shows the outline of the Sulfonylurea (SU) Challenge study explored in the SAD Study (30 to 60 mg of Compound 1).

FIG. 5a shows the effects of placebo (graph a), FIG. 5b shows the effects of 30 mg of Compound 1, and FIG. 5c shows the effects of 60 mg of Compound 1 on glucose infusion rate in the sulfonylurea challenge study. In each graph, Day −2 (baseline) is the top curve and Day 1 (treated) is the bottom curve. The solid line in the curves is the mean value, and the shaded area is the SEM.

FIG. 6a shows the effects of placebo, FIG. 6b shows 30 mg of Compound 1, and FIG. 6c shows 60 mg of Compound 1 on plasma glucose in the sulfonylurea challenge study. In each graph, Day −2 (baseline) is the bottom curve and Day 1 (treated) is the top curve. The solid line in the curves is the mean value, and the shaded area is the SEM.

FIG. 7 shows the MAD study pharmacokinetic results (30 to 120 mg of Compound 1 QD for 10 days).

FIG. 8a shows the MAD study results of the dose response on fasting glucose, FIG. 8b shows the MAD study results of the dose response on fasting insulin, and FIG. 8c shows the MAD study results of the dose response on fasting C-peptide (30 to 120 mg of Compound 1 QD for 10 days).

FIG. 9a shows the effects of placebo, FIG. 9b shows the effects of 30 mg of Compound 1 QD for 10 days, FIG. 9c shows the effects of 60 mg QD for 10 days, and FIG. 9d shows the effects of 120 mg QD for 10 days of Compound 1 on glucose infusion rate in the sulfonylurea challenge study. In each graph, Day −2 (baseline) is the top curve and Day 10 (treated) is the bottom curve. The solid line in the curves is the mean value, and the shaded area is the SEM.

FIG. 10a shows the effects of placebo, FIG. 10b shows the effects of 30 mg of Compound 1 QD for 10 days, FIG. 10c shows the effects of 60 mg QD for 10 days, and FIG. 10d shows the effects of 120 mg QD for 10 days of Compound 1 on plasma glucose in the sulfonylurea challenge study. In each graph, Day −2 (baseline) is the bottom curve and Day 10 (treated) is the top curve. The solid line in the curves is the mean value, and the shaded area is the SEM.

### DETAILED DESCRIPTION

Congenital hyperinsulinism (HI) is the most common cause of recurrent hypoglycemia in infants and children. Estimates of the incidence is approximately 1:30,000 in the United States, but can range from 1:2,500 to 1:50,000 depending on the global region and the rate of consanguinity. Hypoglycemia is often present at birth, but symptoms in neonates can be subtle, leading to delayed or missed diagnosis, placing these patients at high risk of permanent brain injury and even death. Current treatment options are limited, have intolerable side effects, and are not universally effective in all patients; thus, there is an urgent medical need to develop novel therapies for neonates, infants, and children with congenital HI.

Under physiological conditions, blood glucose levels and insulin secretion from the pancreatic β-cells are tightly coupled such that when blood glucose is low insulin production is suppressed. Genetic mutations in β-cell signaling pathways or insulin secretion machinery underlie congenital HI, such that the pancreatic β-cells continue insulin production even in the setting of low blood sugar. Excess insulin not only lowers blood glucose, but also suppresses normal counter-regulatory adaptations to hypoglycemia, suppressing ketogenesis, lipolysis, and gluconeogenesis, leading to a hypoketotic hypoglycemia with complete lack of fuel for the brain. As such, patients with congenital HI are especially vulnerable to neurocognitive consequences, with nearly 50% of patients suffering from developmental defects. Although the severity of congenital HI resolves with time and is very uncommon in older adolescents and adults, the permanent impact of HI during the neonatal and infant period on neurodevelopmental outcomes is seen regardless, underscoring this critical window for brain growth and development and the urgency of treating and preventing hypoketotic hypoglycemia early in life.

For many subjects with HI, the entire pancreas is affected (i.e., diffuse HI), resulting in dysregulated insulin levels with excess secretion of insulin from β-cells. However, paternally inherited mutations of the K_{ATP} channel, may result in a focal region of abnormal β-cells while the remainder of the pancreas is normal (i.e., focal HI).

Hypoglycemic symptoms in the neonate and infant can range in severity and may be difficult to detect. The most severe episodes may present with apnea, seizures, and coma, but symptoms such as lethargy and irritability may be extremely mild. Clinical features such as macrosomia and difficulty feeding may not be present in all neonates affected by congenital HI; thus, a heightened suspicion is required for timely diagnosis and treatment.

Hypoglycemia persisting after 48 hours of life should trigger assessment of congenital HI, among other causes of hypoglycemia. Biochemical diagnosis of congenital HI includes a comprehensive assessment of biomarkers of excess insulin and/or excess insulin action at the time of hypoglycemia (i.e., blood glucose <50 mg/dL). A detectable insulin level at the time of hypoglycemia is highly specific for congenital HI but is not always seen due to sporadic secretion and rapid clearance of insulin; elevated C-peptide is a more robust biomarker. Insulin is the only hormone that can suppress ketogenesis, lipolysis, and gluconeogenesis. Thus, documentation of excess insulin action at the time of hypoglycemia including suppressed ketones, suppressed free fatty acids, and inappropriate glycemic rise in response to glucagon administration are more sensitive markers than isolated insulin levels.

Once HI is diagnosed based on biomarkers, a therapeutic trial of diazoxide is initiated. Subsequent diagnostic steps in those failing to maintain euglycemia on diazoxide may include genetic testing, and if indicated, specialized imaging studies including use of ¹⁸fluorine dihydroxyphenylalanine positron emission tomography (¹⁸F DOPA PET) scan to visualize focal areas of β-cell adenomatosis. While genetic testing is becoming more widespread, it is not done in all patients nor is it required for the diagnosis of congenital HI.

Early diagnosis of congenital HI and effective treatment are essential to prevent life-threatening and devastating neurological sequelae. The hypoketotic nature of the hypoglycemia is critically dangerous as the brain has no source of fuel, and if left untreated has much more severe consequences than other hypoglycemic disorders. Thus, the treatment goal of congenital HI is to rapidly correct hypoglycemia and maintain plasma glucose ≥70 mg/dL, a target higher than in neonates with hypoglycemic disorders where ketones are present.

Treatment is accomplished either through pharmacological therapy or with surgery. Selection of treatment options is highly dependent on clinical severity and there is no option that is universally effective for all patients. In addition, all currently available options (medical and surgical) have major limitations as described below, leaving patients with congenital HI in need of new treatments.

The treatment of pediatric congenital HI is extremely intense; however, this disease is rarely seen in adults. Two studies have documented the clinical course of patients with K_{ATP} mutations that avoided surgeries through the use of complex medical regimens and demonstrated that there is disease resolution in childhood. As discussed, in more than 50% of patients undergoing a near total pancreatectomy in infancy suffer from persistent hypoglycemia that may managed with aggressive and complex medical regimens. By late childhood, however, the hypoglycemia has resolved and nearly all patients have progressed to hyperglycemia requiring insulin therapy.

Those rare patients who do have hypoglycemia persisting into adulthood tend to have mutations in glutamate dehydrogenase (GLUD1) or glucokinase (GCK), each representing less than 4% to 5% of the patient population. Hypoglycemia is often mild, responsive to lower doses of diazoxide and or dietary modifications, and therefore not representative of the congenital HI in children, infants, and neonates who have the most unmet need.

### Compound 1

Compound 1 is a potent small molecule SSTR5 agonist (EC50<1 nM) that is selective over other human SST receptor subtypes, and displays >500-fold greater selectivity for SSTR5 over SSTR2.

Compound 1 refers to 4-[(3S)-3-aminopyrrolidin-1-yl]-6-cyano-5-(3,5-difluorophenyl)-N-[(2S)-1,1,1-trifluoropropan-2-yl]pyridine-3-carboxamide, which has the chemical structure shown below.

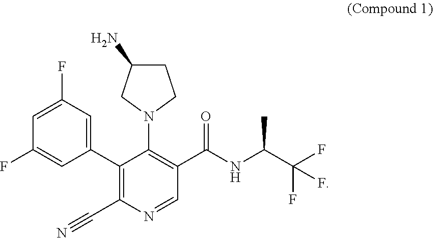 [SMILES: C[C@H](NC(=O)c1cnc(C#N)c(-c2cc(F)cc(F)c2)c1N1CC[C@H](N)C1)C(F)(F)F]

Compound 1 is also referred to as 4-((S)-3-aminopyrrolidin-1-yl)-6-cyano-5-(3,5-difluorophenyl)-N-((S)-1,1,1-trifluoropropan-2-yl)nicotinamide.

### Methods of Treatment

In certain aspects, disclosed herein is a method of reducing endogenous insulin levels in a human comprising administering to the human in need thereof Compound 1, or a pharmaceutically acceptable salt, or solvate thereof. In others aspects, disclosed herein is a method of inhibiting insulin secretion from the pancreas in a human comprising administering to the human in need thereof Compound 1, or a pharmaceutically acceptable salt, or solvate thereof. In some embodiments, the human has congenital hyperinsulinism (HI). In some embodiments, the human has hyperinsulinemic hypoglycemia.

Hyperinsulinemic hypoglycemia describes the condition and effects of low blood glucose caused by excessive insulin. Hypoglycemia due to excess insulin is the most common type of serious hypoglycemia. It can be due to endogenous or exogenous (i.e. injected/administered) insulin. In hyperinsulinemic hypoglycemia there is dysregulation of insulin secretion from pancreatic β-cells. Insulin secretion becomes inappropriate for the level of blood glucose leading to severe hypoglycemia. Hyperinsulinemic hypoglycemia is associated with a high risk of brain injury because insulin inhibits lipolysis and ketogenesis thus preventing the generation of alternative brain substrates (such as ketone bodies). Hence, hyperinsulinemic hypoglycemia must be diagnosed as soon as possible and the management instituted appropriately to prevent brain damage.

In certain aspects, disclosed herein is a method of treating recurrent hypoglycemia in a human with HI, the method comprising administering to the human in need thereof, Compound 1, or a pharmaceutically acceptable salt, or solvate thereof. In certain aspects, disclosed herein is a method of decreasing insulin levels in a human with recurrent hypoglycemia, the method comprising administering to the human in need thereof, Compound 1, or a pharmaceutically acceptable salt, or solvate thereof. In certain aspects, disclosed herein is a method of preventing hypoglycemia in a human with congenital HI, the method comprising administering to the human in need thereof, a compound having the structure of Compound 1, or a pharmaceutically acceptable salt, or solvate thereof.

In some embodiments, the methods disclosed herein comprise a method of treating a recurrent hypoglycemia. In some embodiments, the recurrent hypoglycemia comprises HI. In some embodiments, the recurrent hypoglycemia comprises hypoglycemia due to endogenous insulin, drug induced HI, or hypoglycemia due to exogenous insulin. In some embodiments, the recurrent hypoglycemia comprises congenital HI.

In some embodiments, treating the recurrent hypoglycemia comprises reducing hypoketotic hypoglycemia, lethargy, irritability, vision loss, neurocognitive defects, seizures, apnea, coma, death, or a combination thereof. In some embodiments, treating the recurrent hypoglycemia comprises reducing the risk of brain damage, reducing the extent of brain damage, reducing the risk of pancreatectomy, or a combination thereof. In some embodiments, treating the recurrent hypoglycemia comprises reducing hypokinetic hypoglycemia. In some embodiments, treating the recurrent hypoglycemia comprises reducing lethargy. In some embodiments, treating the recurrent hypoglycemia comprises reducing irritability. In some embodiments, treating the recurrent hypoglycemia comprises reducing vision loss. In some embodiments, treating the recurrent hypoglycemia comprises reducing neurocognitive defects. In some embodiments, treating the recurrent hypoglycemia comprises reducing the risk or brain damage. In some embodiments, treating the recurrent hypoglycemia comprises reducing the extent of brain damage. In some embodiments, treating the hypoglycemia comprises reducing the risk of pancreatectomy. In some embodiments, treating the recurrent hypoglycemia comprises reducing seizures. In some embodiments, treating the recurrent hypoglycemia comprises reducing apnea. In some embodiments, treating the recurrent hypoglycemia comprises reducing the risk of, incidences of, and/or duration of a coma. In some embodiments, treating the recurrent hypoglycemia comprises reducing the risk of death resulting from recurrent and/or prolonged hypoglycemia.

In some embodiments, the methods described herein comprise increasing levels of at least one metabolite. In some embodiments, the metabolite comprises plasma glucose, βhydroxybutyrate, glucagon, or a combination thereof. In some embodiments, the metabolite is increased compared to the level of the metabolite in the subject before treatment. In some embodiments, the metabolite is increased by at least about 1%, 2%, 3%, 4%, 5%, 6%, 7%, 8%, 9%, 10%, 15%, 20%, 25%, 30% or more than 30%.

In some embodiments, described herein is a method of increasing blood glucose levels in a subject with recurrent hypoglycemia to an average blood glucose level of a human without hypoglycemia. In some embodiments, the plasma glucose in a subject with recurrent hypoglycemia is increased to an average level of plasma glucose in a subject without hypoglycemia. In some embodiments, the plasma glucose is increased compared to the level of a subject without hypoglycemia. In some embodiments, a subject with hypoglycemia has a plasma glucose level of less than 70 mg/dL. In some embodiments, a subject without hypoglycemia has a plasma glucose level of more than 70 mg/dL. In some embodiments, the increase in levels of plasma glucose is determined by an intravenous glucose tolerance test.

In some embodiments, the methods described herein comprise decreasing levels of at least one metabolite. In some embodiments, the metabolite comprises insulin, C peptide, or a combination thereof. In some embodiments, the metabolite is decreased compared to the level of the metabolite in the subject before treatment. In some embodiments, the metabolite is decreased by at least about 1%, 2%, 3%, 4%, 5%, 6%, 7%, 8%, 9%, 10%, 15%, 20%, 25%, 30% or more than 30%.

In some embodiments, the methods described herein comprises reducing the level of insulin. In some embodiments, the methods comprise reducing levels of incretin-induced insulin secretion. In some embodiments, the methods comprise reducing insulin secretion from a pancreatic β-cell. In some embodiments, the levels of insulin are decreased as determined by an intravenous glucose tolerance test. In some embodiments, the levels of insulin are decreased by at least about 1%, 2%, 3%, 4%, 5%, 6%, 7%, 8%, 9%, 10%, 15%, 20%, 25%, 30% or more than 30%. In some embodiments, the levels of insulin are decreased compared to the levels of insulin before treatment. In some embodiments, described herein is a method of inhibiting insulin secretion from the pancreas in a human with congenital HI.

In some embodiments, the methods disclosed herein comprise decreasing or inhibiting insulin secretion and minimizing or avoiding glucagon suppression.

### Subjects

In some embodiments, the subject is a human. In some embodiments, the subject is less than 12 years, less than 11 years, less than 10 years, less than 9 years, less than 8 years, less than 7 years, less than 6 years, less than 5 years, less than 4 years, less than 3 years, less than 2 years, or less than 1 year old. In some embodiments, the subject is less than 12 months, less than 11 months, less than 10 months, less than 9 months, less than 8 months, less than 7 months, less than 6 months, less than 5 months, less than 4 months, less than 3 months, less than 2 months, or less than 1 month old.

In some embodiments, the subject has transient neonatal HI. In some embodiments, the subject has persistent neonatal HI (e.g. congenital HI). In some embodiments, the subject has focal HI. In some embodiments, the subject has diffuse HI.

In some embodiments, the subject has at least one mutation in the adenosine triphosphate-dependent potassium (K_{ATP}) channel.

The K_{ATP} form of HI is a genetic disease and its genetic cause is due to defects in either of two genes that make up the potassium channel (called K_{ATP} channel) in the insulin secreting beta-cells of the pancreas. These two genes are the SUR1 gene and the Kir6.2 gene. Normally, when the beta cell senses that glucose levels are elevated, glucose metabolism generates ATP within the beta cell, closing the K_{ATP} channel; resultant beta-cell depolarization initiates the insulin secretion pathway. When the K_{ATP} channel is defective, inappropriate insulin secretion occurs and causes hypoglycemia. Two forms of K_{ATP}-HI exist: diffuse K_{ATP}-HI and focal K_{ATP}-HI. When these mutations are inherited in an autosomal recessive manner (one mutation in the gene inherited from each parent, neither of whom is affected) they cause diffuse disease, meaning every beta-cell in the pancreas is abnormal. Less commonly, autosomal dominant mutations (a mutation in a single copy of the gene) may cause diffuse disease. When a loss of heterozygosity (inheritance of a mutation from the father and loss of the mother's good gene from a few cells in the pancreas) occurs, a focal lesion arises. Abnormal beta cells are limited to this focal lesion and are surrounded by normal beta-cells.

Focal and diffuse K_{ATP}-HI are clinically indistinguishable. Hypoglycemia presents within the first few days of life and often requires large amounts of glucose provided intravenously to keep their blood glucose normal. They may have seizures due to hypoglycemia. Due to the mutation in the K_{ATP} channel, diazoxide is often an ineffective treatment for these children as the mechanism of diazoxide is to act on the K_{ATP} channel. Octreotide, a somatostatin receptor 2 (SST2) agonist, given by injection every 6 to 8 hours or by continuous infusion may be successful (sometimes only in the short term). Glucagon may be given by intravenous infusion to stabilize the blood sugar as a temporary measure. In specialized centers, surgical therapy may be an option. With the recent discovery of diffuse and focal K_{ATP}-HI, attempts to differentiate these two forms by genetic testing and diagnostic imaging (F-DOPA PET scans) are very important: surgical therapy will often cure focal HI, but diffuse HI treated with a sub-total pancreatectomy often fails to completely resolve the disease.

Glutamate dehydrogenase hyperinsulinism (GDH-HI) is another form of congenital HI. It is also known as Hyperinsulinism/Hyperammonemia Syndrome (HI/HA), leucine-sensitive hypoglycemia, and diazoxide-sensitive hypoglycemia. GDH-HI is caused by a mutation in the enzyme glutamate dehydrogenase (GDH). It is inherited in either an autosomal dominant manner or may arise as a sporadically new mutation in a child with no family history. GDH plays an important role in regulating insulin secretion stimulated by amino acids (especially leucine). Individuals with GDH-HI develop hypoglycemia after eating a high protein meal and is often associated with seizure disorders. GDH-HI is also associated with elevated blood concentrations of ammonia, which is derived from protein. Patients with GDH-HI often present later than K_{ATP} channel HI, typically, not until three to four months of age, while others do not have recognizable hypoglycemia until they sleep overnight without a middle of the night feed or after they start higher protein-containing solid foods. The frequency of hypoglycemia is usually less than that associated with K_{ATP}-HI. In addition, GDH-HI can be successfully treated with diazoxide and the avoidance of pure protein loads. Most children with GDH-HI will do very well once recognized, but if the diagnosis is delayed, they may also suffer brain damage from untreated hypoglycemia.

Another form of congenital HI is caused by mutations of the enzyme glucokinase (GK). This defect is inherited in an autosomal dominant fashion but can also arise sporadically. Glucokinase is the “glucose sensor ” for the beta-cell. It tells the beta-cell how high the blood glucose is and when to secrete insulin. Glucokinase mutations that cause HI instruct the beta-cell to secrete insulin at a lower blood glucose than is normal. Like GDH-HI, GK-HI can be treated with diazoxide.

In some embodiments, the subject has a mutation in a genetic loci affecting the insulin secretion machinery in the pancreatic β-cells. The most common mutations (approximately 50% to 60%) occur at the ABCC8 and KCNJ11 genes that encode the SUR-1 and Kir6.2 subunits of the K_{ATP} channel. Other HI forms arise from activating mutations in genes encoding glutamate dehydrogenase (GLUD1) and glucokinase (GCK), each accounting for less than 4% to 5% of the overall HI population. More rare causes include gene mutations in hepatic nuclear transcription factor 4A (HNF4A), hepatic nuclear transcription factor 1A (HNF1A), hexokinase (HK1), uncoupling protein 2 (UCP2), short-chain 3-OH acyl-CoA dehydrogenase (HADH), and solute carrier family 16 member 1 (SLC16A1) encoding monocarboxylate transporter 1 (MCT1). In some embodiments, the human comprises at least one mutation in the ABCC8 and KCNJ11 genes that encode the SUR-1 and Kir6.2 (potassium channel) subunits of the K_{ATP} channel, glutamate dehydrogenase (GLUD1), glucokinase (GCK), hepatic nuclear transcription factor 4A (HNF4A), hepatic nuclear transcription factor 1A (HNF1A), hexokinase (HK1), uncoupling protein 2 (UCP2), short-chain 3-OH acyl-CoA dehydrogenase (HADH), solute carrier family 16 member 1 (SLC16A1), monocarboxylate transporter 1 (MCT1), or a combination thereof.

In some embodiments, the subject has at least one mutation or defect in the ABCC8 and KCNJ11 genes that encode the SUR-1 and Kir6.2 (potassium channel) subunits of the K_{ATP} channel, glutamate dehydrogenase (GLUD1), glucokinase (GCK), hepatic nuclear transcription factor 4A (HNF4A), hepatic nuclear transcription factor 1A (HNF1A), hexokinase (HK1), uncoupling protein 2 (UCP2), short-chain 3-OH acyl-CoA dehydrogenase (HADH), solute carrier family 16 member 1 (SLC16A1), monocarboxylate transporter 1 (MCT1), or a combination thereof.

In some embodiments, the subject has a glucokinase gain-of-function mutation, hyperammonemic HI (glutamate dehydrogenase gain-of-function mutations), short chain acyl coenzyme A dehydrogenase deficiency, carbohydrate-deficient glycoprotein syndrome (Jaeken's Disease), or Beckwith-Wiedemann syndrome, one of the syndromic forms of HI.

In some embodiments, the subject has diazoxide-unresponsive HI.

In some embodiments, the subject has acquired HI. Acquired HI comprises pancreatic insulinoma, nesidioblastosis, drug induced HI, or combinations thereof.

Drug induced HI is caused by drugs that include, but are not limited to, sulfonylurea drugs, aspirin, pentamidine, quinine, disopyramide, bordetella pertussis vaccine or infection, and D-chiro-inositol and myo-inositol

In some embodiments, the subject has failed to respond to a previous treatment for hypoglycemia.

### Diazoxide

In some embodiments, the previous treatment comprises diazoxide. Diazoxide is an oral medication that opens K_{ATP} channels, leading to the inhibition of insulin secretion, and is the only FDA approved treatment for hyperinsulinemic hypoglycemia. Due to the mechanism of action, diazoxide is generally ineffective in patients with mutations in the K_{ATP} channel, rendering nearly half of the congenital HI population “diazoxide non-responsive.” Other genetic forms of congenital HI result from signaling abnormalities upstream of the K_{ATP} channel and are usually responsive to diazoxide.

Failure to respond to diazoxide is indicated by an inability to maintain blood glucose ≥70 mg/dL during an age appropriate fast after 5 days of administration of diazoxide at maximal dose. The 2020 annual report from Congenital HI International Global Registry, the largest congenital HI registry comprised of patient surveys supported by the leading global patient advocacy group, reports that even among those treated with diazoxide and considered responsive, 30% of patients continue to have hypoglycemia. In patients resistant to diazoxide, other medications such as octreotide and enteral dextrose, as well as surgical management, are considered.

Even in patients responsive to diazoxide who are able to attain euglycemia, the side effects may be intolerable. The most common side effects include severe hypertrichosis (approximately 52%), fluid retention (approximately 30%) requiring co-administration of diuretics, and gastrointestinal side effects including feeding aversion (approximately 12%) which often needs feeding therapy services to overcome. Severe sodium and fluid retention can lead to cardiovascular complications, and in 2015, the FDA issued a drug safety communication and included a new warning and precaution in diazoxide labeling after identifying pulmonary hypertension in infants and newborns treated with diazoxide. In one study 4.8% of patients treated with diazoxide experienced pulmonary hypertension. Thus, while diazoxide is approved for congenital HI, throughout the medical and patient community there have been calls for a “better diazoxide”: an oral therapy that prevents hypoglycemia but is also universally effective in all patients regardless of genetic etiology.

### Somatostatin Receptor 2 Agonists

In some embodiments, the previous treatment comprises a somatostatin receptor 2 agonist. In some embodiments, the previous treatment comprises octreotide. Octreotide is a short-acting somatostatin analog which activates the somatostatin receptor type 2 (SST2) independent of the K_{ATP} channel to inhibit insulin secretion. Octreotide is administered by subcutaneous injections multiple times a day and is used off-label in the treatment of diazoxide non-responsive congenital HI, most often due to mutations in the K_{ATP} channel. The glycemic response to octreotide is variable and is also prone to tachyphylaxis or desensitization which may occur after 2 to 3 doses. Octreotide for chronic use is therefore only used effectively in 5% to 10% of the patient population, and co-administration with enteral dextrose is often required. The use of octreotide is associated with risk of gallstones, growth hormone and thyroid hormone suppression, and in rare cases necrotizing enterocolitis, requiring close monitoring and limiting its use in neonates and infants.

The off-label use of monthly long-acting SST2 agonists, lanreotide depot and octreotide long-acting release (LAR), has replaced short-acting octreotide in some patients. Patients receiving long-acting SST2 agonists have variable glycemic profile across the month, with hyperglycemia sometimes lasting for days after the dose, and hypoglycemia often occurring prior to the next scheduled injection. While there is some use in infants, long acting SST2 agonists cannot be withdrawn in the event of unwanted side effects and are challenging to dose thus limiting its widespread use in the youngest patients.

### Glucagon

In some embodiments, the previous treatment comprises glucagon. Glucagon overcomes the gluconeogenic suppressive effects of insulin and is used as part of the diagnostic evaluation of hypoglycemia. Glucagon has been used off-label as a continuous IV infusion in the emergency treatment and stabilization of patients with congenital HI and can reduce the amount of exogenous IV dextrose required in initial stabilization, useful for patients requiring high concentrations of IV dextrose or those suffering from fluid overload. Common IV formulations of glucagon are subject to fibril and gel formation, requiring frequent monitoring to prevent line obstruction as interruptions can cause profound hypoglycemia. Exogenous IV glucagon administration has also been associated with necrolytic migratory erythema, a characteristic rash classically observed in patients with glucagonomas.

Glucagon remains an outpatient emergency treatment of hypoglycemia for patients with congenital HI but is unstable in solution and must be reconstituted from powder before being delivered intramuscularly. Off-label use of continuous glucagon administered subcutaneously through an insulin pump may been effective in reducing hypoglycemia; however, the occurrence glucagon fibrillation and pump blockages remain, and necrolytic migratory erythema remains a risk, which may limit chronic use in patients. Development of novel formulations of stable glucagon are underway and the long-term safety and efficacy is being evaluated.

### Supplemental Dextrose

In some embodiments, the methods described reduce the requirement for the subject to be treated with supplemental dextrose. In some embodiments, the methods described reduce the frequency that supplemental dextrose needs to be administered.

Feeding difficulties occur in nearly one half of patients and are associated with more severe forms of congenital HI. As such, hypoglycemia should not be treated with force-feeding as this often results in reflux, aspiration, and increased feeding refusal behavior. Intragastric continuous dextrose may be useful in severely affected infants to help support blood glucose alone or in combination with other therapies. Given the high rate of post-surgical hypoglycemia, a gastrostomy tube is often placed at the time of a sub-total pancreatectomy in anticipation of a need for long-term dextrose support. The duration of enteral dextrose in combination with other therapy is determined by the efficacy and frequency of those medications.

While enteral dextrose does allow for maintenance of euglycemia in some patients, rates greater than 10 mg dextrose/kg/minute delivers a high osmotic load to the gut and may be associated with poor absorption and intolerance. Adverse events such as vomiting and diarrhea are quite common, and hypoglycemia may still occur in as many as 31% of patients. Pump malfunctions and disconnections result in dramatic hypoglycemia, and significant challenges maintaining normal oral feeding behaviors often remain.

### Surgical Management

In some embodiments, the methods described herein reduce the likelihood of a subject needing surgical management of the recurrent hypoglycemia. In some embodiments, the methods described herein delay the need for surgical management of the recurrent hypoglycemia. Surgical removal of a portion or the entire pancreas may be necessary for patients with congenital HI who are refractory to aggressive medical management. These patients may have a focal region of the pancreas affected or the entire organ diseased, but diffuse and focal forms of HI are clinically indistinguishable. Genetic testing is not required for the diagnosis of congenital HI, but is often performed after a patient has failed diazoxide therapy to identify K_{ATP} mutations and the mode of inheritance. Paternally inherited K_{ATP} mutations suggest focal disease is more likely, with the assumption that there is maternal loss of the normal allele in specific areas of the pancreas. This can be supported by specialized pancreatic imaging with ¹⁸F-DOPA-PET scans where focal uptake of radioactivity may allow visualization of diseased pancreas, but the gold standard for diagnosis of focal versus diffuse HI is through histological examination of intra-operative biopsies.

Approximately 50% of patients with K_{ATP} mutations have focal HI which may be cured with resection of the focus of β-cell adenomatosis. Patients with diffuse HI refractory to medical management often require a near total pancreatectomy (95% to 98%) where a small portion of the pancreas near the duodenum is maintained to preserve the common bile duct. Unfortunately, hypoglycemia remains in more than half of patients following near total pancreatectomies, requiring continued pharmacologic therapy and in severe cases, subsequent surgeries. By adolescence, however, 90% to 100% of patients with near-total pancreatectomies will progress to diabetes and insulin therapy by early adolescence.

### Dosage and Administration

In one embodiment, Compound 1, or a pharmaceutically acceptable salt, or solvate thereof, are used in the preparation of medicaments for the treatment of a recurrent hypoglycemia in a human with hyperinsulinism (HI). Methods for treating any of the diseases or conditions described herein in a subject in need of such treatment, involves administration of pharmaceutical compositions that include at least Compound 1 or a pharmaceutically acceptable salt, active metabolite, prodrug, or pharmaceutically acceptable solvate thereof, in therapeutically effective amounts to said subject.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a subject, who previously experienced at least one symptom of the disease being treated and is currently in remission, a pharmaceutical composition comprising Compound 1, or a pharmaceutically acceptable salt, or solvate thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses of Compound 1 employed for adult human treatment are typically in the range of 0.01 mg-200 mg per day. In one embodiment, the desired dose is conveniently presented in a single dose or in divided doses administered simultaneously or at appropriate intervals, for example as two, three, four or more sub-doses per day.

In some embodiments, Compound 1, or a pharmaceutically acceptable salt, or solvate thereof, is administered in an amount equivalent to about 0.05 mg to about 200 mg of Compound 1. In some embodiments, Compound 1, or a pharmaceutically acceptable salt, or solvate thereof, is administered in an amount equivalent to about 0.5 mg to about 100 mg of Compound 1. In some embodiments, Compound 1, or a pharmaceutically acceptable salt, or solvate thereof, is administered in an amount equivalent to about 1 mg to about 50 mg of Compound 1.

In some embodiments, Compound 1, or a pharmaceutically acceptable salt, or solvate thereof, is administered in amount equivalent to about: 0.05 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13.0 mg, 13.5 mg, 14.0 mg, 14.5 mg, 15.0 mg, 15.5 mg, 16.0 mg, 16.5 mg, 17.0 mg, 17.5 mg, 18.0 mg, 18.5 mg, 19.0 mg, 19.5 mg, or 20.0 mg of Compound 1.

In some embodiments, Compound 1, or a pharmaceutically acceptable salt, or solvate thereof, is administered in a dose of at least about: 0.05 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13.0 mg, 13.5 mg, 14.0 mg, 14.5 mg, 15.0 mg, 15.5 mg, 16.0 mg, 16.5 mg, 17.0 mg, 17.5 mg, 18.0 mg, 18.5 mg, 19.0 mg, 19.5 mg, or 20.0 mg of Compound 1.

In some embodiments, Compound 1, or a pharmaceutically acceptable salt, or solvate thereof, is administered in a dose of no more than about: 0.05 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13.0 mg, 13.5 mg, 14.0 mg, 14.5 mg, 15.0 mg, 15.5 mg, 16.0 mg, 16.5 mg, 17.0 mg, 17.5 mg, 18.0 mg, 18.5 mg, 19.0 mg, 19.5 mg, or 20.0 mg of Compound 1.

In one embodiment, the daily dosages appropriate for the compound of Compound 1, or a pharmaceutically acceptable salt, or solvate thereof, described herein are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

In some embodiments, Compound 1, or a pharmaceutically acceptable salt, or solvate thereof, is administered in a daily dosage equivalent to about 0.01 mg/kg to about 50 mg/kg of Compound 1. In some embodiments, Compound 1, or a pharmaceutically acceptable salt, or solvate thereof, is administered in a daily dosage equivalent to about 0.05 mg/kg to about 10 mg/kg of Compound 1. In some embodiments, Compound 1, or a pharmaceutically acceptable salt, or solvate thereof, is administered in a daily dosage equivalent to about 0.1 mg/kg to about 5.0 mg/kg of Compound 1. In some embodiments, Compound 1, or a pharmaceutically acceptable salt, or solvate thereof, is administered in a daily dosage equivalent to about 0.5 mg/kg to about 2.0 mg/kg of Compound 1.

In some embodiments, Compound 1, or a pharmaceutically acceptable salt, or solvate thereof, is administered in a daily dosage equivalent to about: 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.25 mg/kg, 2.5 mg/kg,2.75 mg/kg, 3.0 mg/kg, 3.25 mg/kg, 3.5 mg, 3.75 mg/kg, 4.0 mg/kg, 4.25 mg/kg, 4.5 mg/kg, 4.75 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, or 10.0 mg/kg of Compound 1.

In some embodiments, Compound 1, or a pharmaceutically acceptable salt, or solvate thereof, is administered in a daily dosage of at least about: 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.25 mg/kg, 2.5 mg/kg,2.75 mg/kg, 3.0 mg/kg, 3.25 mg/kg, 3.5 mg, 3.75 mg/kg, 4.0 mg/kg, 4.25 mg/kg, 4.5 mg/kg, 4.75 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, or 10.0 mg/kg of Compound 1.

In some embodiments Compound 1, or a pharmaceutically acceptable salt, or solvate thereof, is administered in a daily dosage of no more than about: 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.25 mg/kg, 2.5 mg/kg,2.75 mg/kg, 3.0 mg/kg, 3.25 mg/kg, 3.5 mg, 3.75 mg/kg, 4.0 mg/kg, 4.25 mg/kg, 4.5 mg/kg, 4.75 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, or 10.0 mg/kg of Compound 1.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in subjects, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of Compound 1, or a pharmaceutically acceptable salt, or solvate thereof, is: (a) systemically administered to the subject; and/or (b) administered orally to the subject; and/or (c) intravenously administered to the subject. In some embodiments, Compound 1, or a pharmaceutically acceptable salt, or solvate thereof, is administered orally.

In any of the aforementioned aspects are further embodiments comprising single administrations of the effective amount of the compound, including further embodiments in which (i) the compound is administered once a day; or (ii) the compound is administered to the subject multiple times over the span of one day. In some embodiments, Compound 1, or a pharmaceutically acceptable salt, or solvate thereof, is administered daily.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the subject every 8 hours; (iv) the compound is administered to the subject every 12 hours; (v) the compound is administered to the subject every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

### Combination Treatments

In certain instances, it is appropriate to administer at least Compound 1, or a pharmaceutically acceptable salt, or solvate thereof, in combination with one or more other therapeutic agents.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, Compound 1, or a pharmaceutically acceptable salt, or solvate thereof, is co-administered with a second therapeutic agent, wherein the compound of Compound 1, or a pharmaceutically acceptable salt, or solvate thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply be additive of the two therapeutic agents or the patient experiences a synergistic benefit.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

Compound 1, or a pharmaceutically acceptable salt, or solvate thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include oral administration.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

It should be understood that in addition to the ingredients particularly mentioned above, the compounds and compositions described herein may include other agents conventional in the art having regard to the type of formulation in question, for example those suitable for oral administration may include flavoring agents.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used in the specification and claims, the singular forms “a”, “an” and “the” include plural references unless the context clearly dictates otherwise. For example, the term “a sample” includes a plurality of samples, including mixtures thereof.

As used herein, the term “about” a number refers to that number plus or minus 10% of that number. The term “about” a range refers to that range minus 10% of its lowest value and plus 10% of its greatest value.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound of Formula (I), or a pharmaceutically acceptable salt thereof, and a co-agent, are both administered to a patient simultaneously in the form of a single entity or dosage. The term “non-fixed combination” means that the active ingredients, e.g. a compound of Formula (I), or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “article of manufacture” and “kit” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The section headings used herein are for organizational purposes only and are not to be construed as limiting the subject matter described.

### EXAMPLES

The following examples are included for illustrative purposes only and are not intended to limit the scope of the invention.

### Example 1: SSTR Assays

### Membrane Preparation

Crude membrane fractions are prepared from Chinese hamster ovary (CHO) cells stably expressing one of the five human or rodent somatostatin receptor subtypes. The cells are grown to 85-100% confluence on standard tissue culture dishes in DM-MEM growth media (Gibco) with following additives: 10% fetal bovine serum (Gibco), 100 U/mL penicillin (Gibco), 100 ug/mL streptomycin (Gibco), 10 mM HEPES (Gibco), 0.5 mg/mL G-418 (Gibco). To prepare membranes, cells are washed once with 1× Dulbecco's phosphate buffered saline (Gibco) containing 10 mM HEPES (Gibco) then once with sodium free binding buffer (50 mM Tris Base, 5 mM MgCl₂-6H₂O and 1 mM EGTA adjusted to pH 7.8). The cells are then scraped into binding buffer containing a protease inhibitor cocktail (100 ug/mL pepstatin A (Sigma), 50 ug/mL leupeptin (Sigma), 25 ug/mL aprotinin (Sigma) and 10 mg/mL Bacitracin (USB Corporation)). The cells are centrifuged at 43,500 × g, homogenized, and the resulting membranes are collected by centrifugation at 67,000 × g. The membranes are then resuspended in binding buffer containing the protease inhibitor cocktail using a glass dounce homogenizer.

### Functional Assays

General overview: All five SSTR subtypes are Gi coupled G-protein coupled receptors (GPCRs) that lead to decreases in intracellular cyclic AMP (cAMP) when activated by an agonist. Therefore, measurement of intracellular cAMP levels can be used to assess whether compounds of the invention are agonists of SSTR subtypes (John Kelly, Troy Stevens, W. Joseph Thompson, and Roland Seifert, Current Protocols in Pharmacology, 2005, 2.2.1-2.2). One example of an intracellular cAMP assay is described below.

### cAMP Assay Protocol for SST2R:

Four days prior to the assay, 5,000 Chinese hamster ovary cells (CHO-K1, ATCC #CCL-61) stably expressing the human somatostatin receptor subtype 2 are plated in each well of a 96-well tissue culture-treated plate in Ham's F12 growth media (ThermoFisher #10-080-CM) supplemented with 10% donor bovine serum (Gemini Bio-Products #100-506), 100 U/mL penicillin; 100 ug/mL streptomycin; 2 mM L-glutamine (Gemini Bio-Products #400-110) and 0.2 mg/mL hygromycin B (GoldBio #31282-04-9). The cells are cultured at 37° C., 5% CO₂ and 95% humidity. On the day of the assay, the media is aspirated and the cells are treated with 50 μL of 1.6 μM NKH477 (Sigma #N3290) plus various dilutions of compounds of the invention in assay buffer [1× Hank's Balanced Salt Solution (ThermoFisher #SH3058802), 0.5 mM HEPES pH 7.4, 0.1% bovine serum albumin, 0.2 mM 3-Isobutyl-1-methylxanthine (IBMX, VWR #200002-790)]. The cells are incubated for 20 minutes at 37° C. (the final concentration of the compounds of the invention are typically 0-10,000 nM). The cells are treated with 50 μL of lysis buffer (HRTF cAMP kit, Cisbio). The lysate is transferred to 384-well plates and cAMP detection and visualization antibodies are added and incubated for 1-24 hours at room temperature. The time-resolved fluorescent signal is read with a Tecan M1000Pro multiplate reader. The intracellular cAMP concentrations are calculated by regression to a standard curve and are plotted vs. the concentration of the compounds of the invention and the EC₅₀ of the compounds are calculated using standard methods. All data manipulations are in GraphPad Prism v6 or v7.

### cAMP Assay Protocol for SST5R

Four days prior to the assay, 2,000 Chinese hamster ovary cells (CHO-K1, ATCC #CCL-61) stably expressing the human somatostatin receptor subtype 5 are plated in each well of a 96-well tissue culture-treated plate in Ham's F12 growth media (ThermoFisher #10-080-CM) supplemented with 10% donor bovine serum (Gemini Bio-Products #100-506), 100 U/mL penicillin; 100 μg/mL streptomycin; 2 mM L-glutamine (Gemini Bio-Products #400-110) and 0.25 mg/mL G418 Sulfate (GoldBio #108321-42-2). The cells are cultured at 37° C., 5% CO2 and 95% humidity. On the day of the assay, the media is aspirated and the cells are treated with 50 μL of 1.6 μM NKH477 (Sigma #N3290) plus various dilutions of compounds of the invention in assay buffer [1× Hank's Balanced Salt Solution (ThermoFisher #SH3058802), 0.5 mM HEPES pH 7.4, 0.1% bovine serum albumin, 0.2 mM 3-Isobutyl-1-methylxanthine (IBMX, VWR #200002-790)]. The cells are incubated for 20 minutes at 37° C. (the final concentration of the compounds of the invention are typically 0-10,000 nM). The cells are treated with 50 μL of lysis buffer (HRTF cAMP kit, Cisbio) for 30 minutes and then the lysate is diluted to 250 μL with assay buffer. The lysate is transferred to 384-well plates and cAMP detection and visualization antibodies are added and incubated for 1-24 hours at room temperature. The time-resolved fluorescent signal is read with a Tecan M1000Pro multiplate reader. The intracellular cAMP concentrations are calculated by regression to a standard curve and are plotted vs. the concentration of the compounds of the invention and the EC₅₀ of the compounds are calculated using standard methods. All data manipulations are in GraphPad Prism v6 or v7.

Illustrative biological activity of compounds is demonstrated in the following Tables, by evaluating the inhibition of cAMP activities via human SST receptors.

Table 1 demonstrates illustrative biological selectivity of Compound 1 for the SST5 receptor over SST2 receptor, by evaluating the inhibition of cAMP activities via human SST5 receptor and human SST2 receptor.

TABLE 1
Illustrative SST5 Activity and Selectivity Data
Demonstrating Preference for SST5 vs SST2

| SST5 potency | SST2 potency | Fold selectivity for |
|---|---|---|
| (EC₅₀; nM) | (EC₅₀; nM) | SST5 vs SST2 |
| <1 | >100 nM | >500 |

### Example 2: Liver Microsomal Stability Assay Protocol:

The in vitro stabilities of compounds of interest were determined for various species using pooled male and female human, pooled male Sprague-Dawley rat, pooled male Cynomolgus monkey, and pooled male Beagle dog liver microsomes at microsomal protein concentrations of 0.5 mg/mL. Incubations were carried out in a potassium phosphate buffer (50 mM). The NADPH-generating system was composed of NADP+ (1 mM), magnesium chloride (3 mM), EDTA (1 mM), glucose-6-phosphate (5 mM) and glucose-6-phosphate dehydrogenase (1 Unit/mL) for all experiments. Compounds of interest in DMSO/acetonitrile were added to achieve a final incubation concentration of 1 μM (final DMSO content was 0.1% v/v and final acetonitrile content was 0.9%). The final incubation volume was 400 μL. Incubations were conducted at 37° C. for 0, 5, 10, 20, 40 and 60 minutes in a shaking water bath and terminated by removing 50 μL of incubation mixture and adding to 100 μL of ice cold acetonitrile containing internal standard. Following precipitation by centrifugation at 3500 rpm and 4° C. for 30 minutes, compounds of interest and internal standard were analyzed in the resultant supernatant using a multiple reaction monitoring (MRM) LC-MS/MS method. MS conditions were optimized for each analyte. Depletion rates of compounds of interest were measured and half-life, scaled intrinsic clearance, and predicted scaled systemic clearance calculations were made using this data.

### Example 3: Genetic Models of Hyperinsulinism in Rodents

Representative assays evaluating the effect of a selective somatostatin subtype (sst5) agonist described herein in genetic models of hyperinsulinism in rodents, specifically the SUR1^{-/-} mouse model, are described. The SUR1^{-/-} mouse reproduces the key pathophysiological features of K_{ATP} congenital hyperinsulinism (HI), the most common and severe genetic form of HI. SUR1^{-/-} mice are both significantly more hypoglycemic when fasted and significantly more hyperglycemic when glucose-loaded compared with control wild-type. Evaluation of the effect of oral administration of a somatostatin subtype (sst5) agonist described herein on plasma glucose levels after a fast is described below.

### In vivo Experiments

SUR1^{-/-} mice and wild type mice are administered a somatostatin subtype (sst5) agonist described herein at a dose of 30 mg/Kg/day for 1 week. Fasting plasma glucose, insulin, and beta-hydroxybutyrate concentrations are measured after a 16 hr fast at baseline and after 1 week of treatment. Glucose and an insulin tolerance tests are performed during the treatment period.

Sample size: Average fasting plasma glucose levels in SUR1^{-/-} mice are 59.4 +/−5.0 mg/dL. With 5 mice per group there is greater than 90% power to detect a difference of 20% (equivalent to bringing the levels to normal range) on fasting plasma glucose levels in treated versus control-treated SUR1^{-/-} mice (using alpha 0.05).

Treatment groups: (1) a compound described herein; (2) selective somatostatin 2 agonist; and (3) Placebo.

Genotype groups: (1) SUR1^{-/-} mice; and (2) Wild type mice

### Experimental Procedures

Fasting Evaluation: Fasting plasma glucose are measured after a 16 hour fast. Plasma glucose and beta-hydroxybutyrate levels are checked by a hand held glucose meter (Nova Stat Strip glucose meters) in blood obtained from a tail nick (only one nick will be necessary) and 15 microliters of blood are collected to measure insulin levels.

Intraperitoneal glucose tolerance test: After an overnight fast, mice are given an i.p. dose of glucose (2 g/kg). Plasma glucose and insulin concentrations are measured at baseline and every 30 min for 2 hrs. Fifteen microliters of blood/time point is obtained and measured for insulin levels.

Insulin tolerance test: After a 6 hr fast, mice are given an i.p. injection of insulin (1 unit/kg). Glucose concentration is measured at baseline and every 10 min for 30 minutes or until the mice reach a hypoglycemic state, then every 30 min for 2 hrs.

### In Vitro Experiments

The direct effects of a somatostatin subtype (sst5) agonist described herein or selective somatostatin 2 agonist on insulin secretion are tested in isolated pancreatic islets from wild type and SUR1^{-/-} mice. The direct effects of the compounds are also tested in islets isolated from patients with K_{ATP}HI who undergo pancreatectomy and those from healthy human volunteers.

Batch incubation: 5 islets for each well with 4 replicates of each condition in 96-well plate format are used for the study. Islets are exposed to 4 concentrations of glucose (0, 5, 10 and 25 mM) or mixture of amino acids (0, 2, 4 and 10 mM) in the absence or presents of 4 concentrations of 2 compounds (a somatostatin subtype (sst5) agonist described herein, somatostatin 2 agonist). The effects of compounds and the effective doses on insulin secretion are obtained after those experiments.

Cytosolic calcium measurements: Cytosolic calcium ([Ca²⁺]ᵢ) dynamics are assessed using Fura-2 as calcium indicator; islets isolated from wild type or SUR1^{-/-} mice are exposed to glucose and amino acids. The effects of the compounds on [Ca²⁺]ᵢ dynamics are directly evaluated.

Islets perifusion: After batch incubations and calcium measurements, the effective concentration of compounds are determined. The effects of those compounds with effective dose on insulin secretion dynamics are evaluated in perifused islets.

K_{ATP}HI human islets: Compounds are also tested with K_{ATP}HI human islets. Islets are isolated from surgical specimens from patients with K_{ATP}HI who underwent pancreatectomy. K_{ATP}HI are perifused in response to amino acid and glucose stimulation in the absence or presence of the compounds. [Ca²⁺]ᵢ dynamics are also tested.

### Example 4: Human Islet Pharmacology

The activity of Compound 1 on insulin and glucagon secretion in human pancreatic islets from three healthy adult donors (Prodo Labs, Aliso Viejo, Calif.) was evaluated using a perifusion system that enables dynamic insulin and glucagon measurements from the same islet preparation in response to different stimuli. Specifically, the effects on glucose-stimulated and sulfonylurea (tolbutamide)-stimulated insulin secretion and arginine-stimulated glucagon secretion were investigated and compared to those of a maximally effective concentration of diazoxide. Islets were collected in culture media (Prodo Labs #PIM-S001GMP) supplemented with 5% human AB serum (Prodo Labs #PIM-ABS001GMP), 1% glutamine-glutathione (Prodo Labs #PIM-G001GMP) and an antibiotic cocktail of amphotericin B, ciproflaxin and gentamicin (Prodo Labs #PIM-3X001GMP) and plated at a density of 1500-3000 islet equivalents (IEQ) in 10-cm nontreated tissue culture dishes (Thomas Scientific #1182M59). Islets were cultured at 37° C., 5% CO₂ and 95% humidity for 20 h. Dynamic insulin secretion assays were performed using a perifusion system, Peri5 (Biorep Technologies, FL. Glucose (Boston BioProducts #BM-675) at 3 mM (G3) or 16.7 mM (G16.7), or glucose at 16.7 mM plus 100 μM tolbutamide (Sigma #T0891; G16.7+Tolb) were prepared in perifusion buffer, PB=[24 mM NaHCO₃ (Fisher Scientific #S233-500), 120 mM NaCl (Spectrum Chemical #S0155), 4.8 mM KCl (Fisher Scientific #P217-500), 2.5 mM CaCl₂ (Fisher Scientific #C614-500), 1.2 mM MgCl₂ (Sigma #M8266), 10 mM HEPES (Gibco #15630-080) and 0.1% BSA (EMD Millipore #12675-100GM)]. Various concentrations of Compound 1 and diazoxide (Sigma #D9035) were prepared in G16.7 or in G16.7+Tolb. On the day of the assay, 150 IEQ were distributed in each perifusion chamber (Biorep #Peri-chamber). Ten chambers were loaded with a bead suspension (Biorep #Peri-beads-20) followed by 150 IEQ and another layer of bead suspension. Chambers were filled with PB, placed in the perifusion equipment and equilibrated with G3 for 63 min (flow rate=283 μL/min) followed by the perifusion steps for insulin secretion shown in Table 2. Perifusate from the conditions in duplicates were collected in 96 well plates (Fisher Scientific #12-565-368) and stored at −80° C. for insulin analysis. Insulin concentration was quantified using the commercially-available Mercodia Insulin ELISA (Mercodia #10-1113-10) kit. Perfusate samples were diluted in the supplied diabetes sample buffer (Mercodia #10-1195-01) and added to the supplied pre-coated assay plates. Series of reagent additions and washes were performed according to the manufacturer's instructions. The optical density of each well was determined at 450 nm in a Tecan M1000Pro (Tecan, Switzerland) multiplate reader. Insulin concentrations were calculated by regression to the standard curve using GraphPad Prism (GraphPad, San Diego, Calif.).

TABLE 2
Schedule of events for stimulation of insulin secretion
in isolated islets from healthy donors.

| | | | Compound 1 (μM) | | | |
|---|---|---|---|---|---|---|
| | Time | Flow rate | Chamber | Chamber | Chamber | Chamber |
| Condition | (min) | (μL/min) | 1 and 2 | 3 and 4 | 5 and 6 | 7 and 8 |
| G3 | 0-10 | 200 | 0 | 0 | 0 | 0 |
| G16.7 | 11-31 | 100 | 0 | 1 | 0.1 | 0.01 |
| G16.7 + Tolb | 32-45 | 100 | 0 | 1 | 0.1 | 0.01 |
| G3 | 46-59 | 100 | 0 | 0 | 0 | 0 |

For glucagon secretion, glucose (Boston BioProducts #BM-675) solutions, G3, G16.7 and G3 plus 20 mM arginine (Sigma #A8094; G3+Arg) were prepared in PB [24 mM NaHCO₃ (Fisher Scientific #S233-500), 120 mM NaCl (Spectrum Chemical #S0155), 4.8 mM KCl (Fisher Scientific #P217-500), 2.5 mM CaCl₂ (Fisher Scientific #C614-500), 1.2 mM MgCl₂ (Sigma #M8266), 10 mM HEPES (Gibco #15630-080) and 0.1% BSA (EMD Millipore #12-675-100GM)]. Various concentrations of Compound 1 and diazoxide (Sigma #D9035) were prepared in G16.7 or in G3+Arg. On the day of the assay, ten perifusion chambers (Biorep #Peri-chamber) were loaded with a bead suspension (Biorep #Peri-beads-20) followed by 300 IEQ and another layer of bead suspension. Chambers were filled with PB, placed in the perifusion equipment and equilibrated with G3 for 74 min (flow rate=230 μL/min) followed by the perifusion steps for glucagon secretion shown in Table 3. Perifusate from the conditions in duplicates was collected in 96 well plates (Fisher Scientific #12-565-368) and stored at −80° C. for glucagon analysis. Glucagon concentration was quantified using the commercially-available Mercodia Glucagon ELISA (Mercodia #10-1271-01) kit. undiluted perfusate samples were added to the supplied pre-coated assay plates. Series of reagent additions and washes were performed according to the manufacturer's instructions. The optical density of each well was determined at 450 nm in a Tecan M1000Pro (Tecan, Switzerland) multiplate reader. Glucagon concentrations were calculated by regression to the standard curve using GraphPad Prism (GraphPad, San Diego, Calif.).

TABLE 3
Schedule of events for stimulation of glucagon secretion
in isolated islets from healthy donors

| | | | Compound 1 (μM) | | | Diazoxide (μM) | |
|---|---|---|---|---|---|---|---|
| | Time | Flow rate | Chamber | Chamber | Chamber | Chamber | Chamber |
| Condition | (min) | (μL/min) | 1 and 2 | 3 and 4 | 5 and 6 | 7 and 8 | 9 and 10 |
| G3 | 0-10 | 200 | 0 | 0 | 0 | 0 | 0 |
| G3 + Arg | 11-38 | 100 | 0 | 1 | 0.01 | 100 | 10 |
| G16.7 | 39-59 | 100 | 0 | 1 | 0.01 | 100 | 10 |

Human islets responded to the stimulation agents by secreting insulin in a biphasic manner. In response to 16.7 mM glucose (G16.7), the first phase was rapid and short, and was followed by a second phase characterized by a lower insulin secretion rate but was constant and lasted as long as G16.7 was present. Similar to glucose, tolbutamide in the presence of 16.7 mM glucose (G16.7+Tolb) induced a biphasic response. A transition to 3 mM glucose (G3), resulted in the expected decline in insulin secretion. Islets treated with the lowest Compound 1 concentration (0.01 μM) exhibited responses to both glucose and tolbutamide that were similar to those exposed to control. Compound 1 at 1 μM had the strongest inhibitory effect on insulin during both phases with both stimuli, while Compound 1 at 0.1 μM showed an intermediate effect.

Compound 1 inhibited insulin secretion in response to an increase to 16.7 mM glucose as well as to tolbutamide in the presence of 16.7 mM glucose (G16.7+Tolb) in a concentration dependent manner in islets from three healthy adult donors. Glucagon secretion from pancreatic α-cells is calcium dependent and biphasic, but the initial phase is slightly faster than with insulin secretion due to a higher number of readily releasable glucagon granules. In addition, the second phase is marked by an increasing acceleration of the exocytotic rate as a result of faster translocation of granules from the interior to release sites. Compound 1 had a small non-concentration dependent inhibitory effect on arginine-stimulated glucagon secretion (up to 24%) compared to diazoxide which suppressed glucagon secretion up to 57% in islets from a healthy donor.

### Example 5: Hyperinsulinemic Hypoglycemia Model in Rats

Sulfonylurea drugs, such as glyburide, inhibit K_{ATP} channels and can be used in rat preclinical models to mimic the disease state of congenital HI patients with K_{ATP} channel mutations. Administration of sulfonylurea drugs have been shown to stimulate pancreatic secretion of insulin preclinically in ex vivo and in vivo animal models, as well as clinically in humans. This increase in insulin results in a dramatic decline in blood glucose that can last for hours following a single dose or persistent lowering of blood glucose if administered repeatedly, mimicking the chronic hypoglycemia observed with congenital HI and allowing for assessment of efficacy of potential treatments acutely and over days.

This model of glyburide-induced hyperinsulinemic hypoglycemia was used to assess the pharmacodynamic effects of Compound 1 after a single administration in male and female adult rats and male neonatal rats.

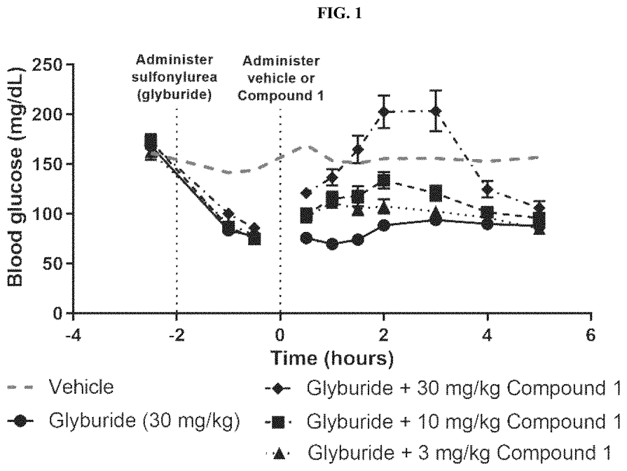

Glyburide was administered to raise insulin levels and induce hypoglycemia in adult male Sprague Dawley rats. Oral administration of 30 mg/kg glyburide reduced blood glucose levels from mean baseline levels of 168 mg/dL to 70 mg/dL in male rats as seen in as seen in FIG. 1.

Two hours following glyburide administration, Compound 1 was given by oral gavage and blood glucose was monitored over 5 hours. Oral administration of Compound 1 dose-dependently increased blood glucose levels compared to administration of vehicle. In the male rats, blood glucose levels reached up to 111, 134, and 203 mg/dL with 3, 10, and 30 mg/kg Compound 1, respectively, compared to 94 mg/dL with vehicle treatment over the 5-hour time period. Significant increases in the cumulative blood glucose response over 5 hours represented as the area under the curve (AUC) of the response were seen with administration of ≥10 mg/kg Compound 1. The 10 mg/kg minimum effective dose of Compound 1 in this model corresponds to a Cmax of 45 ng/mL and an AUC of 590 ng·h/mL in non-fasted male rats.

### Example 6: A Clinical Trial to Evaluate Safety, Pharmacokinetics, and Pharmacodynamics of Compound 1

A non-limiting example of a clinical trial of a SST5 agonist in humans is described below.

### Purpose

This Phase 1, first-in-human, double-blind, randomized, placebo-controlled study will evaluate the safety of Compound 1 in healthy volunteers as well as the relationship between exposure and key pharmacodynamic (PD) parameters. The study will initiate with a single ascending dose (SAD) phase followed by a multiple ascending dose (MAD) phase. Food effect will also be evaluated in subset of subjects in the SAD phase. Results will be used to inform endpoints and other parameters in later studies in patients with congenital HI.

### Intervention

Screening Period: 28 days

Treatment Period: 1 day/single dose in the SAD; 10 days in the MAD

Assessment and Observation: 6, 7, and 10 days (5 days in each arm) in SADs 1a, 1b, and 1c, respectively; 17 days in the MAD

Follow-up Period: 7 and 10 days after the last dose of study drug in the SADs and MAD, respectively (Day 8/SAD and Day 20/MAD)

SAD 1a/Part 1a: ascending single dose, randomized, placebo-controlled, double-blind; up to 10 cohorts, n=8/cohort; subjects in SAD 1a will undergo an IVGTT. Doses will escalate from 0.5 mg to 120 mg.

SAD 1b/Part 1b: ascending single dose, randomized, placebo-controlled, double-blind; up to 3 cohorts, n=8/cohort; subjects in SAD 1b will undergo an SU challenge and euglycemic clamp procedure. Doses are 30 mg or 60 mg.

SAD 1c/Part 1c: single dose, 1-arm crossover food effect investigation; 1 cohort, n=6; SAD 1c will not be placebo-controlled or blinded. The dose is 120 mg.

MAD/Part 2: multiple ascending dose, randomized, placebo-controlled, double-blind; up to 5 cohorts, n=9/cohort; subjects in MAD will receive Compound 1 or the placebo. Subjects will undergo a mixed meal tolerance test (MMTT) and an SU challenge and euglycemic clamp procedure. The doses will not exceed the doses in the SAD. Doses will escalate from 30 mg to 120 mg.

### Inclusion Criteria

Healthy male and female subjects ≥18 to ≤55 years of age, at time of Screening. Females must be either: postmenopausal for at least 12 months or surgically sterile; or agree to use a stable and permitted highly effective method of contraception from Screening until at least 30 days after the last dose of study drug. Males must be surgically sterile; or remain abstinent; or agree to use a spermicide-coated condom when sexually active with a female partner of childbearing potential from Screening until at least 30 days after the last dose of study drug. The female partner should also use a highly effective form of contraception during this same time period. Male subjects must also agree to not donate sperm for the duration of the study and until at least 90 days after the last dose of study drug.

In overall good health according to age (medical history, physical examination, vital signs, body mass index [BMI] of 18.5 to 28 kg/m², and laboratory assessments), as judged by the Investigator at Screening.

### Exclusion Criteria

Subjects will be excluded based on the following criteria: females who are pregnant or lactating; prior treatment with Compound 1; use of prohibited prescribed or non-prescribed medications and/or non-medications/alternative medicinal products within 7 days prior to Screening and is not willing to forego use of these substances during the study (unless otherwise specified); use of medications that are strong inducers of cytochrome P450 CYP3A4 within 30 days prior Screening of this trial or strong inhibitors of CYP3A4 within 14 days prior to Screening; use of any investigational drug within the past 60 days or 5 half-lives, whichever is longer, prior to Screening; fasting plasma glucose (PG) >100 mg/dL at Screening; any condition that in the opinion of the Investigator would jeopardize the subject's appropriate participation in this study.

### Outcome Measures

To evaluate the safety and tolerability of single and multiple doses of Compound 1.

To evaluate the PK of single and multiple doses of Compound 1.

To determine the effect of food (high-fat meal) on the PK of Compound 1.

To assess the PD effect of Compound 1 on plasma glucose (PG), insulin, C peptide; and when applicable, β-hydroxybutyrate, glucagon, gastric inhibitory peptide (GIP), and glucagon-like peptide-1 (GLP-1), levels under basal and stimulated conditions (intravenous glucose tolerance test [IVGTT], mixed meal tolerance test [MMTT], sulfonylurea [SU] induced hypoglycemia).

Patients with congenital HI have fasting hypoglycemia due to excess endogenous insulin secretion. Metabolic biomarkers including glucose, insulin, C-peptide, and βhydroxybutyrate will be collected throughout both the SAD and MAD to assess the effect of single and multiple doses of Compound 1.

The ability of Compound 1 to suppress endogenous insulin secretion will also be analyzed utilizing stimulatory tests including IVGTT (SAD Part 1a), MMTT (MAD), and SU-induced insulin secretion (SAD Part 1b, MAD).

### IV Glucose Tolerance test (IVGTT)

In SAD 1a, subjects will undergo an IVGTT to demonstrate the ability of Compound 1 to suppress endogenous insulin and increase blood glucose concentration in a stimulated setting in which they are provided intravenous glucose. The results from the IVGTT will help inform selection of the single dose that will be used in SAD 1b and SAD 1c and also to inform the doses used in the MAD.

A summary of the procedure is as follows: Collect pre-IV bolus blood samples for measurement of PG, insulin, and C-peptide. At 1 hour after study drug dosing/Time 60 (±30 minutes) or nominal time, administer an IV bolus of glucose at 300 mg/kg in 20% solution, within 60 seconds (use the most recent body weight measurement for glucose dose). Collect post-IV bolus blood samples for measurement of PG, insulin, and C-peptide.

### Sulfonylurea (Glibenclamide) Challenge and Euglycemic Clamp

In SAD 1b and the MAD, subjects will undergo an SU challenge in the setting of a euglycemic clamp procedure. Sulfonylurea drugs act to close K_{ATP} channel of the pancreatic β-cell, stimulating endogenous insulin secretion. Given that the majority (50%) of patients with congenital HI harbor a mutation in the K_{ATP} channel, the SU challenge models this genetic form of HI in healthy volunteers. Intravenous glucose will be used during the euglycemic clamp to maintain glucose within a target range. In the presence of an SU, the amount of intravenous (IV) glucose required to maintain a target glucose will increase, similar to patients with congenital HI who require supplemental glucose. The SU challenge cohorts in SAD 1b will assess the ability of a single dose of Compound 1 to reduce or prevent SU-induced glucose lowering as measured by reduced IV glucose infusion (reduced glucose “support”). IV glucose requirements will be assessed as a glucose infusion rate, or GIR (mg dextrose/kg body weight/minute). In the MAD, the durability of the Compound 1 effect will be measured by assessing the GIR required to maintain euglycemia during the SU challenge after 10 days of Compound 1 dosing compared to the baseline SU challenge in the absence (predose) of Compound 1.

Glibenclamide will be used for SU-induced hypoglycemia. Based on the available dose-response data in healthy volunteers, 5 mg of glibenclamide will be orally administered to healthy volunteers in selected single- and multi-dose cohorts. The risk of hypoglycemia due to overdose is negligible because continuous glucose monitoring by a clamp device will be performed for each subject and IV infusion of glucose will be provided to maintain each subject's baseline glucose levels.

Glibenclamide is eliminated by metabolism by CYP2C9 (with a minor contribution from CYP3A4). Compound 1 showed little or no potential for inhibition or induction of the major human CYP enzymes including those involved in glibenclamide metabolism. Therefore, Compound 1 is not expected to inhibit the clearance of glibenclamide.

Glibenclamide reaches peak systemic concentrations (tₘₐₓ) within 2 to 6 hours after glibenclamide dosing under fasting conditions with a half-life of approximately 10 hours. Glibenclamide will be administered approximately 1 hour prior to administration of Compound 1 and blood samples will be collected before and after glibenclamide administration. The actual timing of the Compound 1 dose administration relative to glibenclamide will be based on observed Compound 1 PK and will be documented in the SRC meeting memo (or other written communication).

The euglycemic glucose clamp will be performed by means of a glucose clamp device (ClampArt®; Profil Institut für Stoffwechselforschung, Neuss, Germany). The subject will be connected to the clamp device that will monitor the subject's PG continuously. The device will calculate aggregated PG values every minute. Based on these values, GIR will be calculated every minute using the algorithm implemented into the device and glucose will be administered automatically by the device to keep the PG concentration of the subject constant at a pre-determined target level. Approximately 2 mL of blood per hour will be sampled for glucose monitoring by ClampArt®/the clamp device. The device's glucose measurements will be verified approximately every 30 minutes or more frequently, if needed, by PG measurements with a laboratory glucose analyzer (Super GL glucose analyzer). Food Effect of a Hi₂h-fat Meal

In SAD 1c, subjects will participate in a 1-arm crossover investigation to evaluate the effect of a high-fat meal on Compound 1 absorption.

### Mixed Meal Tolerance Test (MMTT)

The pancreatic β-cells in congenital HI have been shown to be very sensitive to incretin-induced insulin secretion. An MMTT will be conducted during the MAD study to assess the impact of Compound 1 on incretin-induced insulin secretion. This will be conducted at baseline (before study drug dosing) in the MAD and at Day 6 at which Compound 1 is predicted to be at steady state.

The purpose of the MMTT is to evaluate the effect of Compound 1 on plasma insulin in response to multiple fuel sources (protein, fat) and to evaluate the impact on incretin-induced insulin secretion. Subjects are to fast at least 10 hours overnight until ingestion of the mixed meal (which is to occur approximately 4 hours after study drug dosing), and then restrict further food intake until the end of the MMTT.

Start the MMTT (i.e., drink the mixed meal) on Day −1 and Day 6 at 4 hours (±30 minutes) post-study drug dosing (i.e., Time 240 minutes) or at nominal time. The total duration of the MMTT will be approximately 3.5 hours from when the first pre-drink blood sample is collected until the last MMTT blood sample is collected. The subject is to be in the supine position during the MMTT, except while consuming the mixed meal. The procedure will be as follows:

Collect pre-drink fasting blood samples for measurement of PG, insulin, C-peptide, glucagon, GIP, and GLP-1 at the timepoints specified in (SOA).

At 4 hours after study drug dosing/Time 240 minutes (or nominal time), the subject is to drink 400 mL (2×200-mL bottles) Resource® Energy (Nestlé Health Science) drink (mixed meal) within ≤10 minutes.

Collect post-drink blood samples for measurement of PG, insulin, C-peptide, glucagon, and GLP-1 at the timepoints specified in (SOA). The subject is to not eat or drink (except for water) until the last MMTT blood sample is collected.

Blood samples, except for PG (Super GL), will be analyzed by the central laboratory.

### Results from the SAD STUDY

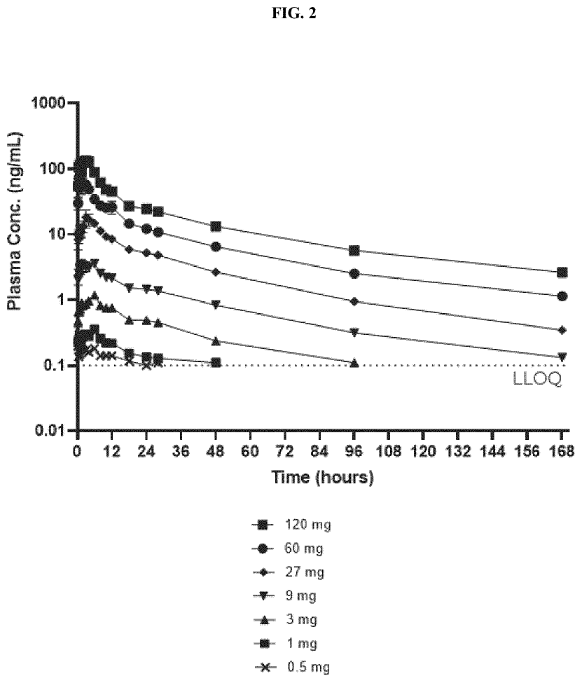

Pharmacokinetic results from the SAD study are shown in FIG. 2. Data shown are mean±SEM. All doses n=6; except n=12 for 60 mg which was evaluated in both IVGTT and sulphonylurea challenge. When 120 mg was administered within 30 minutes of a standard adult high fat breakfast, a significant reduction in exposure was observed. A half-life ˜40 hours was observed and tmax ˜1-2 hours at efficacious doses.

Compound 1 showed oral bioavailability with dose-proportional exposure.

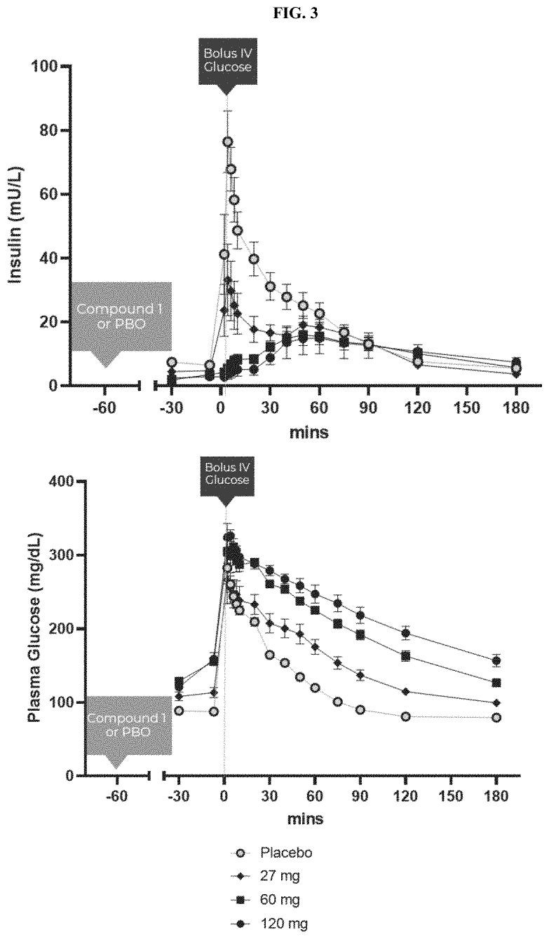

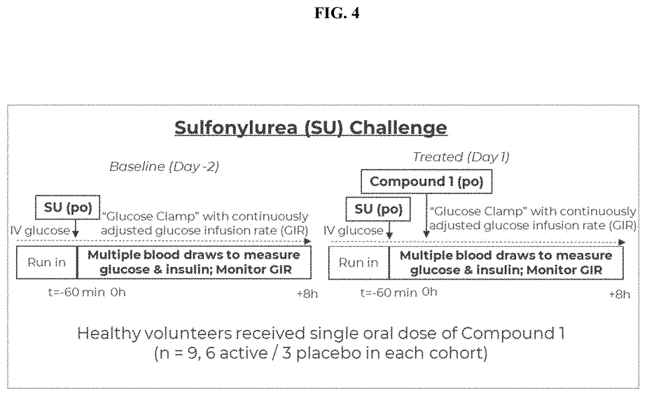

Compound 1 was also evaluated in the context of an intravenous glucose tolerance test (IVGTT), with oral Compound 1 or placebo being administered prior to the intravenous (IV) administration of glucose. In the absence of pharmacologic intervention, IV administration of glucose leads to the secretion of insulin which will result in lowering blood glucose back to baseline values. Results showed that oral administration of Compound 1 prior to the IVGTT dose-dependently suppressed insulin secretion and resulted in a rapid and sustained increase in plasma glucose levels (see FIG. 3). Compound 1 dose-dependently suppressed glucose stimulated insulin secretion. Data shown are mean±SEM. N=6 Compound 1 treated per dose; N=14 placebo. IVGTT=intravenous glucose tolerance test; PBO=placebo.

Compound 1 dose-dependently reduced insulin secretion stimulated by bolus IV glucose (IVGTT) and reduced glucose uptake by tissues resulting in prolonged elevation of plasma glucose. The measured % reduction in insulin AUC was about 22%, about 40%, and about 47%, respectively for the 27 mg, 60 mg, and 120 mg dose.

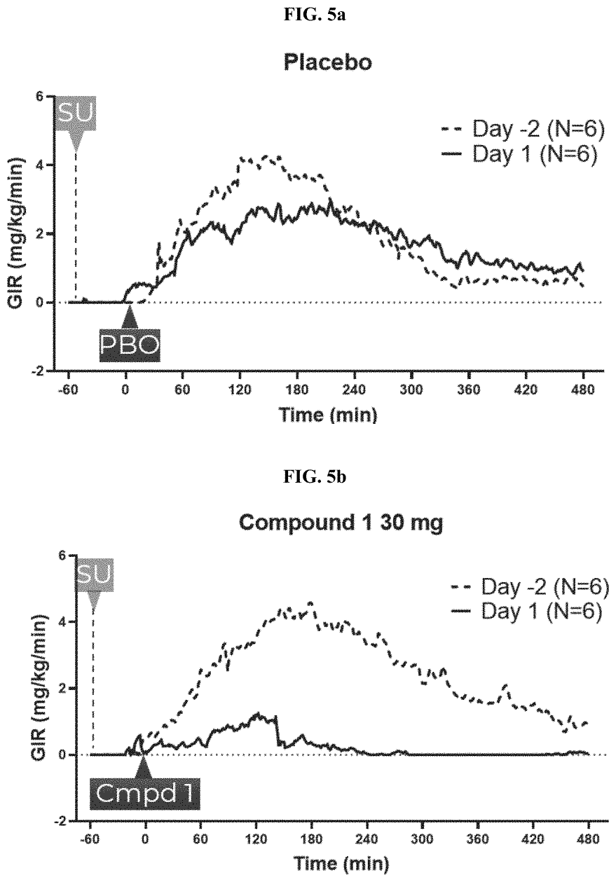

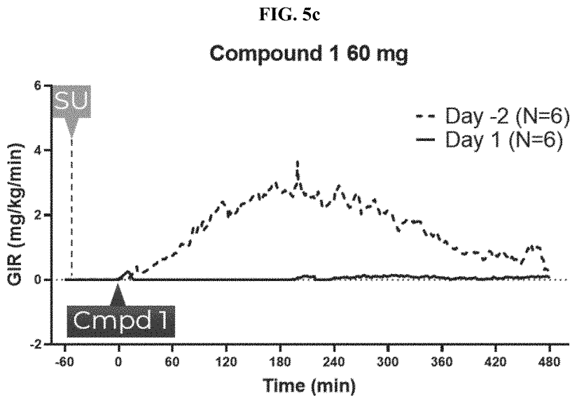

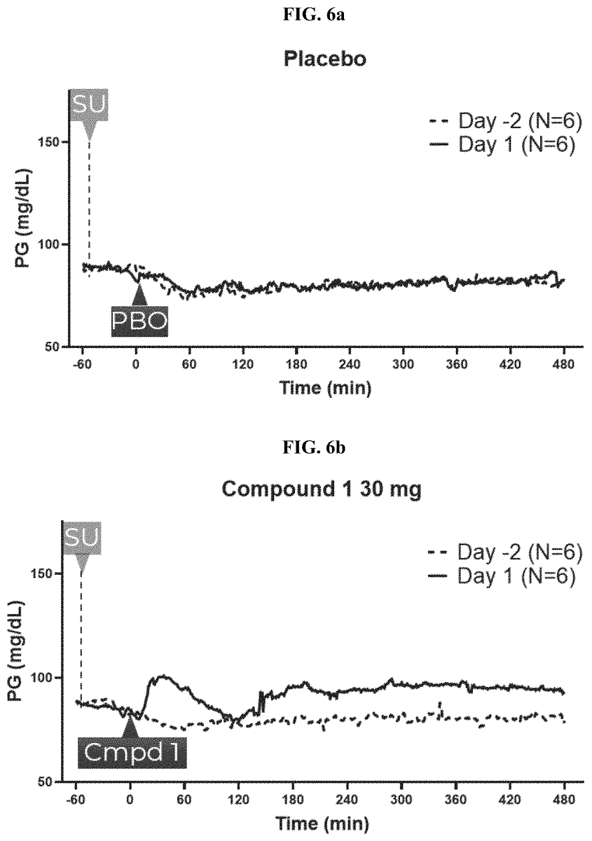

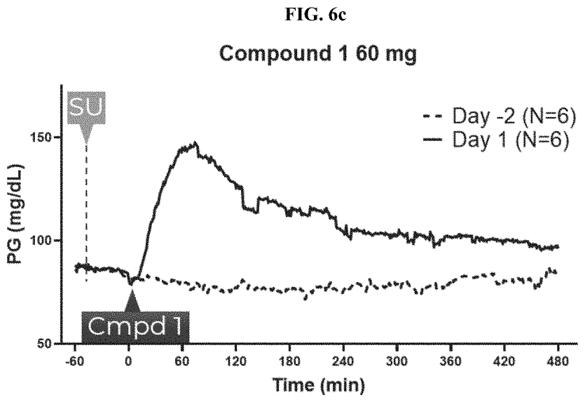

In the sulfonylurea (glibenclamide) challenge and euglycemic clamp model, 30 mg or 60 mg Compound 1 reversed sulfonylurea-induced hyperinsulinism. Results from the SAD cohorts showed that Compound 1 reversed sulfonylurea-induced hyperinsulinism in a dose-dependent fashion within minutes of administration, eliminating the need for IV glucose support for the duration of the study period. Compound 1 eliminated the need for IV glucose support by inhibiting constitutive insulin secretion. (see FIG. 5a, 5b, 5c and FIG. 6a, 6b, 6c).

In this SAD study, Compound 1 demonstrated pharmacologic proof-of-concept for SST5 agonism. Compound 1 provided a dose-dependent reduction in glucose-induced insulin secretion in the intravenous glucose tolerance test. Compound 1 provided a dose-dependent reversal of sulfonylurea-induced insulin secretion achieved in the pharmacologic model of hyperinsulinism.

### Results from the MAD Study

Compound 1 was generally safe and well tolerated after QD dosing at 30, 60, and 120 mg/day for 10 days.

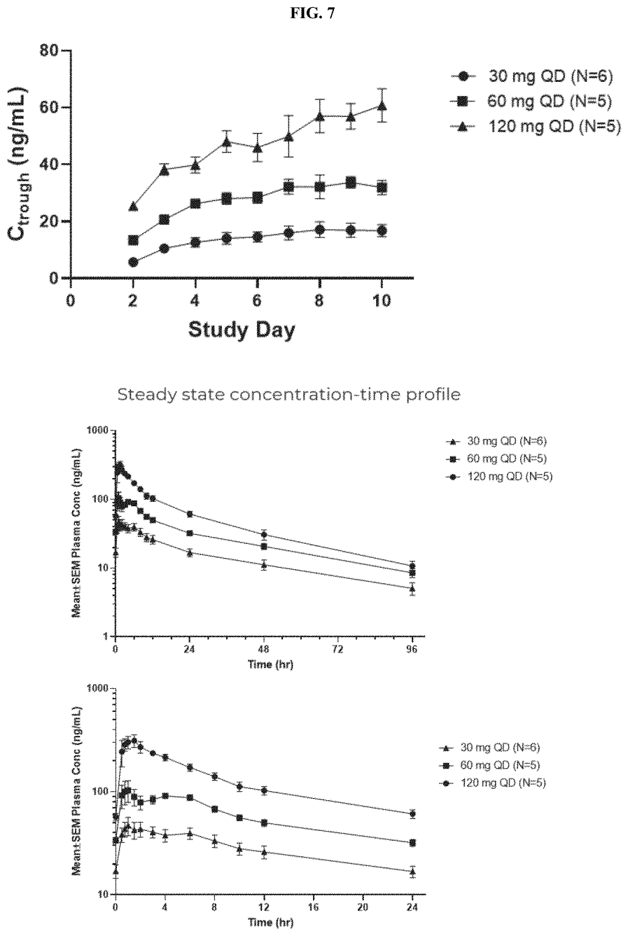

Pharmacokinetic results from the SAD study are shown in FIG. 7. Data shown are trough plasma levels and are displayed as mean±SEM. The single dose PK profile and exposures are comparable to SAD at the same dose (no shown). Steady state trough levels are achieved after ˜8 days of QD dosing. The mean accumulation at steady state was ˜1.6-1.8× for C_{maz} and ˜2-2.5× for AUC. Modestly greater than proportional increases in exposures were observed between 30 and 120 mg doses.

In the mixed meal tolerance test (MMTT), a dose dependent increase in plasma glucose was observed on Day 6 in subjects treated with Compound 1. The increases in AUC were similar between subjects treated with 30 mg and 60 mg doses (˜30%). The increase in plasma glucose AUC in subjects treated with 120 mg doses was ˜60%. There was also more sustained suppression of insulin and C-peptide with increasing dose.

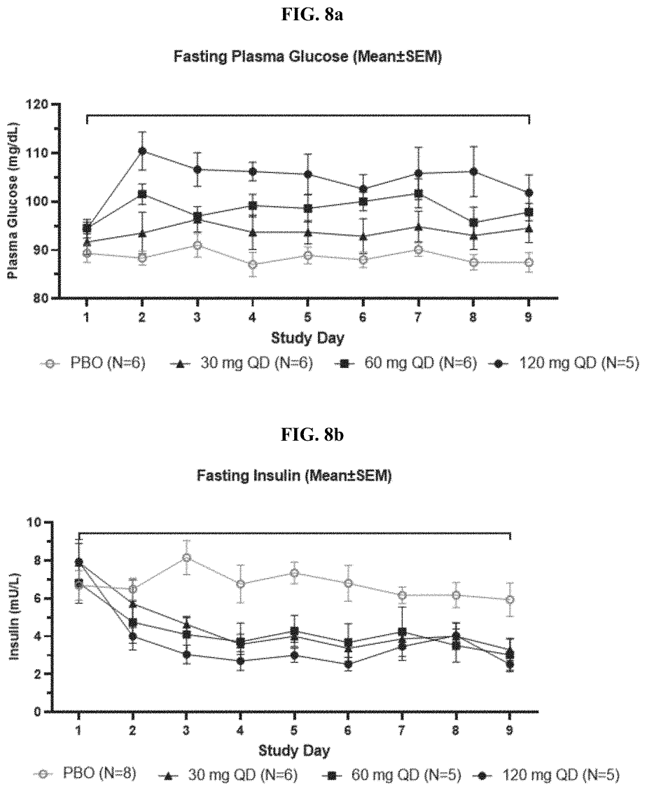

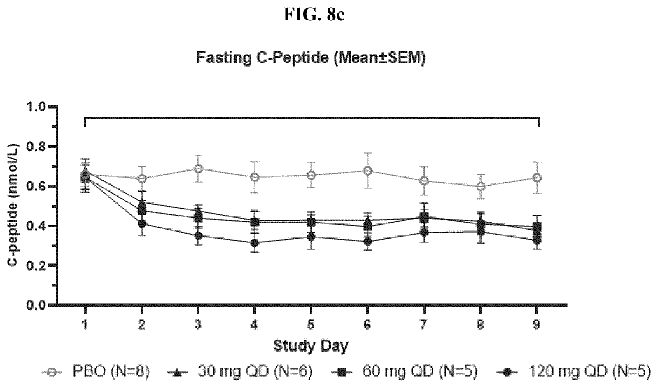

Subjects treated with Compound 1 also revealed a dose-dependent increase in fasting plasma glucose. Fasting insulin and C-peptide levels were also suppressed. However, there was a lack of a clear dose response for fasting insulin, and suppression of C-peptide was much more pronounced at the 120 mg dose when compared to the 30 mg and 60 mg doses. Levels of insulin and C-peptide remained suppressed at 96 h post-dose in the subjects treated with 120 mg dose, but plasma glucose was similar to placebo (see FIG. 8a, FIG. 8b, FIG. 8c).

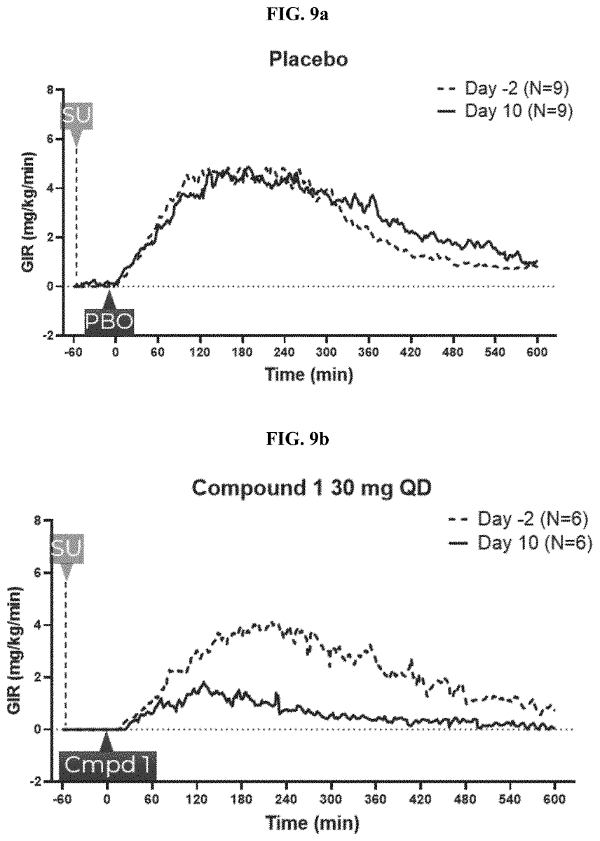

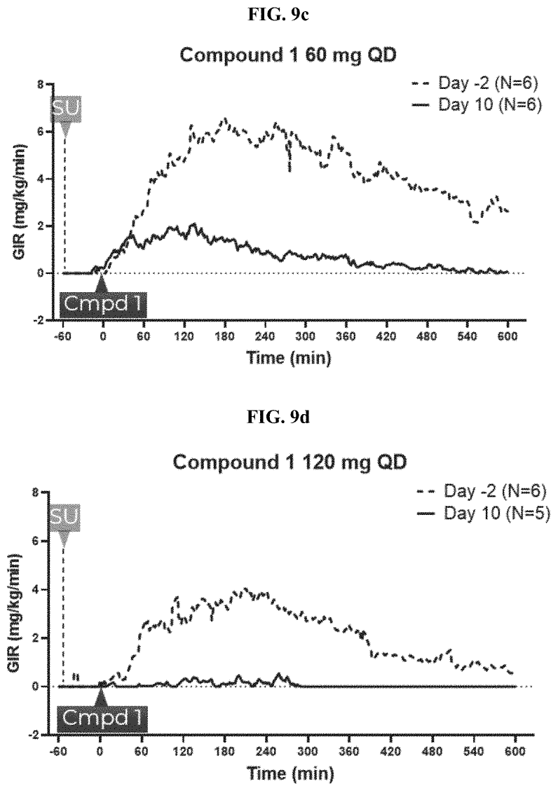

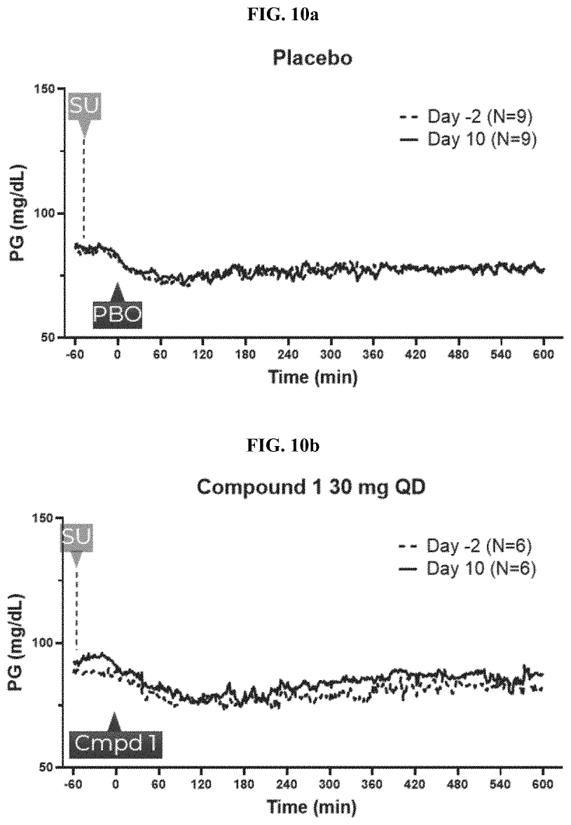

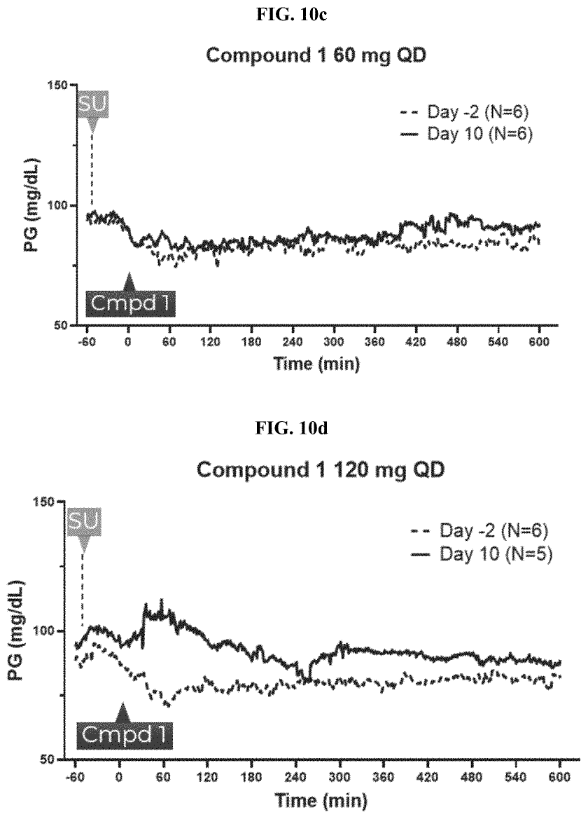

In the sulfonylurea (glibenclamide) challenge and euglycemic clamp model, Compound 1 reversed sulfonylurea-induced hyperinsulinism after 10 days of dosing 30, 60, or 120 mg QD. Results showed that Compound 1 reversed sulfonylurea-induced hyperinsulinism in a dose-dependent fashion within minutes of administration, eliminating the need for IV glucose support. Compound 1 eliminated the need for IV glucose support by inhibiting constitutive insulin secretion. A mild elevation of plasma glucose was observed on in the 120 mg cohort. (see FIG. 9a, FIG. 9b, FIG. 9c, FIG. 9d, and FIG. 10a, FIG. 10b, FIG. 10c, FIG. 10d). Insulin and C-peptide levels were suppressed on day 10 in all cohorts.

While preferred embodiments of the present invention have been shown and described herein, it will be obvious to those skilled in the art that such embodiments are provided by way of example only. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating hyperinsulinism (HI) in a human, the method comprising administering to the human in need thereof a compound having the structure of Compound 1, or a pharmaceutically acceptable salt, or solvate thereof:

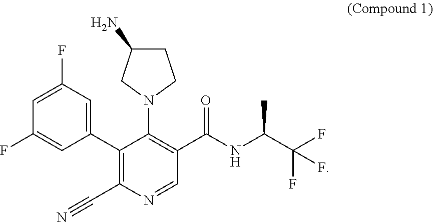 [SMILES: C[C@H](NC(=O)c1cnc(C#N)c(-c2cc(F)cc(F)c2)c1N1CC[C@H](N)C1)C(F)(F)F]

2. The method of claim 1, wherein the hyperinsulinism comprises congenital hyperinsulinism.

3. The method of claim 1, wherein the human comprises at least one mutation in the adenosine triphosphate-dependent potassium (K_{ATP}) channel; or at least one mutation or defect in the ABCC8 and KCNJ11 genes that encode the SUR-1 and Kir6.2 (potassium channel) subunits of the K_{ATP} channel, glutamate dehydrogenase (GLUD1), glucokinase (GCK), hepatic nuclear transcription factor 4A (HNF4A), hepatic nuclear transcription factor 1A (HNF1A), hexokinase (HK1), uncoupling protein 2 (UCP2), short-chain 3-OH acyl-CoA dehydrogenase (HADH), solute carrier family 16 member 1 (SLC16A1), or monocarboxylate transporter 1 (MCT1); or a combination thereof.

4. The method of claim 1, wherein the hyperinsulinism comprises:
transient hyperinsulinism, focal hyperinsulinism, or diffuse hyperinsulinism;
glucokinase gain-of-function mutations, hyperammonemic hyperinsulinism (glutamate dehydrogenase gain-of-function mutations), short chain acyl coenzyme A dehydrogenase deficiency, carbohydrate-deficient glycoprotein syndrome (Jaeken's Disease), or Beckwith-Wiedemann syndrome;
diazoxide-unresponsive hyperinsulinism;
or a combination thereof.

5. The method of claim 1, wherein the hyperinsulinism is acquired hyperinsulinism which comprises pancreatic insulinoma, nesidioblastosis, drug induced hyperinsulinism, or combinations thereof.

6. The method of claim 1, wherein treating hyperinsulinism comprises:
increasing levels of plasma glucose, β-hydroxybutyrate, glucagon, or a combination thereof;
reducing plasma levels of: insulin, C peptide, or a combination thereof;
reducing plasma insulin levels;
reducing levels of incretin-induced insulin secretion;
maintaining plasma glucose levels at above at least 50 mg/dL, above at least 60 mg/dL, above at least 70 mg/dL, or above at least 80 mg/dL;
reducing insulin secretion from pancreatic β-cells;
decreasing or inhibiting insulin secretion and minimizing or avoiding glucagon suppression;
reducing the risk of brain damage, reducing the extent of brain damage, reducing the risk of pancreatectomy, or a combination thereof;
reducing hypoketotic hypoglycemia, reducing lethargy, reducing irritability, reducing the risk of vision loss, reducing the risk of neurocognitive defects, reducing the risk of seizures, reducing the risk of apnea, reducing the risk of coma, reducing the risk of death, or a combination thereof;
or a combination thereof.

7. The method of claim 1, wherein Compound 1, or a pharmaceutically acceptable salt, or solvate thereof, is administered orally once daily or twice daily.

8. The method of claim 7, wherein Compound 1, or a pharmaceutically acceptable salt, or solvate thereof, is administered in amount equivalent to about 0.05 mg to about 200 mg of Compound 1.

9. The method of claim 7, wherein Compound 1, or a pharmaceutically acceptable salt, or solvate thereof, is administered in a daily amount equivalent to about 0.1 mg/kg to about 5.0 mg/kg of Compound 1.

10. The method of claim 7, wherein Compound 1, or a pharmaceutically acceptable salt, or solvate thereof, is administered in a daily amount equivalent to about: 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.25 mg/kg, 2.5 mg/kg, 2.75 mg/kg, 3.0 mg/kg, 3.25 mg/kg, 3.5 mg, 3.75 mg/kg, 4.0 mg/kg, 4.25 mg/kg, 4.5 mg/kg, 4.75 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, or 10.0 mg/kg of Compound 1.

11. The method of claim 7, wherein Compound 1, or a pharmaceutically acceptable salt, or solvate thereof, is administered in a daily amount sufficient to maintain plasma glucose levels at above at least 50 mg/dL, above at least 60 mg/dL, above at least 70 mg/dL, or above at least 80 mg/dL over at least 12 hours, at least 18 hours, or at least 24 hours.

12. A method of treating congenital hyperinsulinism in a human, the method comprising administering to the human in need thereof, a compound having the structure of Compound 1, or a pharmaceutically acceptable salt, or solvate thereof:

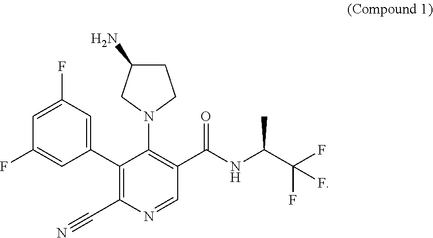 [SMILES: C[C@H](NC(=O)c1cnc(C#N)c(-c2cc(F)cc(F)c2)c1N1CC[C@H](N)C1)C(F)(F)F]

13. The method of claim 12, wherein the human comprises: at least one mutation or defect in the adenosine triphosphate-dependent potassium (K_{ATP}) channel; at least one mutation or defect in the ABCC8 and KCNJ11 genes that encode the SUR-1 and Kir6.2 (potassium channel) subunits of the K_{ATP} channel, glutamate dehydrogenase (GLUD1), glucokinase (GCK), hepatic nuclear transcription factor 4A (HNF4A), hepatic nuclear transcription factor 1A (HNF1A), hexokinase (HK1), uncoupling protein 2 (UCP2), short-chain 3-OH acyl-CoA dehydrogenase (HADH), solute carrier family 16 member 1 (SLC16A1), monocarboxylate transporter 1 (MCT1), or a combination thereof.

14. The method of claim 12, wherein congenital hyperinsulinism comprises:
transient hyperinsulinism, focal hyperinsulinism, or diffuse hyperinsulinism;
glucokinase gain-of-function mutations, hyperammonemic hyperinsulinism (glutamate dehydrogenase gain-of-function mutations), short chain acyl coenzyme A dehydrogenase deficiency, carbohydrate-deficient glycoprotein syndrome (Jaeken's Disease), or Beckwith-Wiedemann syndrome;
diazoxide-unresponsive congenital hyperinsulinism;
or a combination thereof.

15. The method of claim 12, wherein the human is less than 12 years old, less than 6 years old, less than 4 years old, less than 3 years old, less than 2 years old, less than 1.5 years old, less than 1 year old, or less than 6 months old.

16. The method of claim 12, wherein treating congenital hyperinsulinism comprises:
increasing levels of plasma glucose, (3-hydroxybutyrate, glucagon, or a combination thereof;
reducing plasma levels of: insulin, C peptide, or a combination thereof;
reducing plasma insulin levels;
reducing levels of incretin-induced insulin secretion;
maintaining plasma glucose levels at above at least 50 mg/dL, above at least 60 mg/dL, above at least 70 mg/dL, or above at least 80 mg/dL;
reducing insulin secretion from pancreatic β-cells;
decreasing or inhibiting insulin secretion and minimizing or avoiding glucagon suppression;
reducing the risk of brain damage, reducing the extent of brain damage, reducing the risk of pancreatectomy, or a combination thereof;
reducing hypoketotic hypoglycemia, reducing lethargy, reducing irritability, reducing the risk of vision loss, reducing the risk of neurocognitive defects, reducing the risk of seizures, reducing the risk of apnea, reducing the risk of coma, reducing the risk of death, or a combination thereof;
or a combination thereof

17. The method of claim 12, wherein: Compound 1, or a pharmaceutically acceptable salt, or solvate thereof, is administered orally once daily or twice daily at a daily dosage equivalent to about 0.1 mg/kg to about 5.0 mg/kg of Compound 1.

18. The method of claim 17, wherein Compound 1, or a pharmaceutically acceptable salt, or solvate thereof, is administered in a daily amount sufficient to maintain plasma glucose levels at above at least 50 mg/dL, above at least 60 mg/dL, above at least 70 mg/dL, or above at least 80 mg/dL over at least 12 hours, at least 18 hours, or at least 24 hours.

19. A method of inhibiting insulin secretion from the pancreas in a human with congenital hyperinsulinism comprising administering to the human in need thereof a compound having the structure of Compound 1, or a pharmaceutically acceptable salt, or solvate thereof:

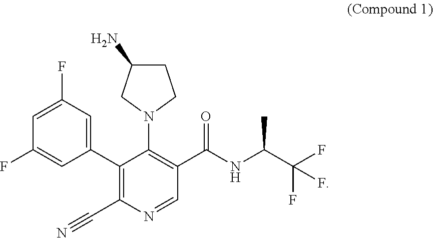 [SMILES: C[C@H](NC(=O)c1cnc(C#N)c(-c2cc(F)cc(F)c2)c1N1CC[C@H](N)C1)C(F)(F)F]

20. The method of claim 19, wherein inhibiting insulin secretion comprises inhibiting insulin secretion from pancreatic β-cells, or inhibiting incretin-induced insulin secretion; and wherein inhibiting insulin secretion comprises treating recurrent hypoglycemia.

21. The method of claim 19, wherein Compound 1, or a pharmaceutically acceptable salt, or solvate thereof, is administered in a daily amount equivalent to about 0.1 mg/kg to about 5.0 mg/kg of Compound 1.

22. A method of decreasing insulin levels in a human with recurrent hypoglycemia, the method comprising administering to the human in need thereof, a compound having the structure of Compound 1, or a pharmaceutically acceptable salt, or solvate thereof:

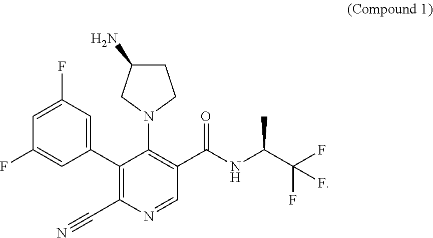 [SMILES: C[C@H](NC(=O)c1cnc(C#N)c(-c2cc(F)cc(F)c2)c1N1CC[C@H](N)C1)C(F)(F)F]

23. The method of claim 22, wherein the human with recurrent hypoglycemia comprises hyperinsulinism; and wherein Compound 1, or a pharmaceutically acceptable salt, or solvate thereof, is administered in a daily amount equivalent to about 0.1 mg/kg to about 5.0 mg/kg of Compound 1.

24. The method of claim 22, wherein decreasing levels of insulin comprises reducing levels of incretin-induced insulin secretion; and wherein Compound 1, or a pharmaceutically acceptable salt, or solvate thereof, is administered in a daily amount equivalent to about 0.1 mg/kg to about 5.0 mg/kg of Compound 1.

25. A method of treating or preventing hypoglycemia in a human with hyperinsulinism, the method comprising administering to the human in need thereof, a compound having the structure of Compound 1, or a pharmaceutically acceptable salt, or solvate thereof:

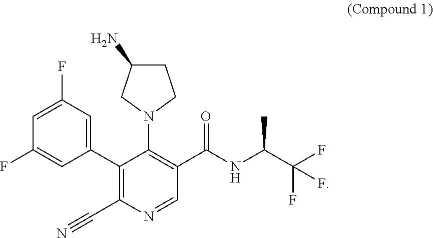 [SMILES: C[C@H](NC(=O)c1cnc(C#N)c(-c2cc(F)cc(F)c2)c1N1CC[C@H](N)C1)C(F)(F)F]

26. The method of claim 25, wherein the hyperinsulinism comprises congenital hyperinsulinism; and wherein Compound 1, or a pharmaceutically acceptable salt, or solvate thereof, is administered daily in an amount equivalent to about 0.1 mg/kg to about 5.0 mg/kg of Compound 1.

27. The method of claim 25, wherein the hypoglycemia comprises recurrent hypoglycemia; and wherein Compound 1, or a pharmaceutically acceptable salt, or solvate thereof, is administered daily in an amount equivalent to about 0.1 mg/kg to about 5.0 mg/kg of Compound 1.

28. The method of claim 25, wherein treating or preventing hypoglycemia comprises:
reducing hypoketotic hypoglycemia, lethargy, irritability, macrosomia, vision loss, neurocognitive defects, seizures, apnea, coma, death, or a combination thereof;
reducing the risk of brain damage, reducing the extent of brain damage, reducing the risk of pancreatectomy, or a combination thereof;
increasing levels of plasma glucose, β-hydroxybutyrate, glucagon, or a combination thereof;
reducing the levels of: insulin, C peptide, or a combination thereof;
reducing plasma levels of insulin;
decreasing or inhibiting insulin secretion and minimizing or avoiding glucagon suppression; or
combinations thereof.

29. The method of claim 25, wherein Compound 1, or a pharmaceutically acceptable salt, or solvate thereof, is administered daily in an amount equivalent to about 0.1 mg/kg to about 5.0 mg/kg of Compound 1; and wherein Compound 1, or a pharmaceutically acceptable salt, or solvate thereof, is administered daily in an amount sufficient to maintain plasma glucose levels at above at least 50 mg/dL, above at least 60 mg/dL, above at least 70 mg/dL, or above at least 80 mg/dL over at least 12 hours, at least 18 hours, or at least 24 hours.
